(19) United States
(12) Patent Application Publication
(10) Pub. No.: US 2011/0189227 A1
(43) Pub. Date: Aug. 4, 2011

(54) PORCINE CIRCOVIRUS TYPE 2 AND USE THEREOF

(75) Inventors: DAE SUB SONG, SUWON-SI (KR); BO KYU KANG, YONGIN-SI (KR); HYE KWON KIM, INCHEON-CITY (KR); BONG KYUN PARK, SUWON-SI (KR)

(73) Assignee: GREEN CROSS VETERINARY PRODUCTS CO., LTD., YESAN-GUN (KR)

(21) Appl. No.: 12/784,910

(22) Filed: May 21, 2010

(30) Foreign Application Priority Data
Feb. 4, 2010 (KR) 10-2010-0010656

(51) Int. Cl. A61K 39/12; C12N 7/00; A61P 31/12

(52) U.S. Cl. 424/204.1; 435/235.1

(57) ABSTRACT

Porcine circovirus type 2 (PCV2) having high replication ability in host cells and use thereof.

### CROSS-REFERENCE TO RELATED APPLICATIONS

This application claims priority under 35 USC 119 (a)-(d) to Korea Application No. 10-2010-0010656, filed on Feb. 4, 2010, in the Korean Intellectual Property Office, the contents of which are hereby incorporated by reference in their entirety

### BACKGROUND OF THE INVENTION

1. Field of the Invention

The present invention relates to a porcine circovirus type 2 and use thereof.

2. Description of the Related Art

Porcine circovirus type 2 (PCV2) is a small, icosahedral, non-enveloped DNA virus having a diameter in the range of 17 to 22 nm with a single-stranded circular genome. PCV2 and porcine circovirus type 1 (PCV1) have a degree of sequence identity of about 80%. While PCV1 is non-pathogenic, PCV2-infected pigs have symptoms such as postweaning multisystemic wasting syndrome (PMWS), porcine respiratory disease complex (PRDC), PCV2-caused enteritis, PCV2-caused reproductive diseases, porcine dermatitis nephritis syndrome (PDNS), atrophy, respiratory symptoms, diarrhea, jaundice, and gastric ulcer. The PCV2 is a member of the family of Circoviridae. The PCV2 genome does not encode for a viral DNA-polymerase, making it dependent on cellular enzymes to complete its infectious cycle.

When PCV2 is inoculated in pigs, a high variation in virus replication is observed. It has been observed that PCV2 is able to replicate better in a host when simultaneously inoculated with other viruses such as porcine reproductive and respiratory syndrome virus (PRRSV) or porcine parvovirus (Allan et al (2000) Arch Virol. 145, 2421-2429, Allan et al (2000) J Vet Med B. 47, 81-94).

The titer of PCV2 present in a cell culture infected by PCV2 is generally in the range of 3.4 to 4.5 log₁₀ TCID₅₀/ml. U.S. Pat. No. 7,566,562 discloses a method for in vitro cultivation of a porcine circovirus, the method including: inoculating cells of a continuous animal cell line in a culture medium with porcine circovirus; cultivating the continuous animal cell line in the medium in the presence of one or more inhibitors of endosomal-lysosomal system acidification; and isolating the porcine circovirus from the medium and/or the infected continuous animal cell line.

However, there is still a need to develop PCV2 having high replication ability in host cells. In particular, there is still a need to develop PCV2 having high replication ability without using a virus replication enhancer such as at least one inhibitor of endosomal-lysosomal system acidification.

### SUMMARY OF THE INVENTION

The present invention provides a porcine circovirus type 2 (PCV2) having high replication ability in a host cell. In one embodiment, the present invention also provides a method of attenuating a PCV2 having high replication ability in a host cell. In another embodiment, the present invention also provides an immunogenic composition including an attenuated PCV2 having high replication ability in a host cell. In a further embodiment, the present invention also provides a method of preventing or treating PCV2 infection-associated diseases in animals by using the immunogenic composition.

According to an aspect of the present invention, there is provided a composition for treating or preventing circovirus infection in animals, the composition comprising at least on attenuated virus selected from the group consisting of attenuated circovirus PCV2 GC0504 and PCV2 GC0505.

According to another aspect of the present invention, there is provided a method of producing attenuated circovirus, the method comprising reacting at least one virus selected from the group consisting of circovirus PCV2 GC0504 and PCV2 GC0505 with formalin to prepare attenuated circovirus.

According to another aspect of the present invention, there is provided a method of treating or preventing circovirus infection in animals, the method comprising administering the composition according to any one of the claims provided herein in an effective amount into the animals.

According to another aspect of the present invention, there is provided a Circovirus PCV2 GC0504 comprising a genome having a nucleotide sequence of SEQ ID NO: 1.

According to another aspect of the present invention, there is provided a Circovirus PCV2 GC0505 comprising a genome having a nucleotide sequence of SEQ ID NO: 4.

### BRIEF DESCRIPTION OF THE DRAWINGS

The above and other features and advantages of the present invention will become more apparent by describing in detail exemplary embodiments thereof with reference to the attached drawings.

FIG. 1 is a graph illustrating results of real time-polymerase chain reaction (PCR) of PCV2 having various concentrations.

FIG. 2 is a graph illustrating PCV2 elution via nasal cavity after PCV2 challenge-inoculation into young pigs.

FIG. 3 is a graph illustrating PCV2 elution via nasal cavity after challenge-inoculation of PCV2 and porcine reproductive and respiratory syndrome virus (PRRSV) into young pigs.

FIG. 4 is a graph illustrating PCV2 elution via excrement after PCV2 challenge-inoculation into young pigs.

FIG. 5 is a graph illustrating PCV2 elution via excrement after challenge-inoculation of PCV2 and PRRSV into young pigs.

### DETAILED DESCRIPTION OF THE INVENTION

Hereinafter, the present invention will now be described more fully with reference to the accompanying drawings, in which exemplary embodiments of the invention are shown.

One or more embodiments of the present invention provide a composition for treating or preventing circovirus infection in animals, the composition including at least one attenuated virus selected from the group consisting of attenuated circoviruses PCV2 GC0504 and PCV2 GC0505.

PCV2 GC0504 may include a genome having a nucleotide sequence of SEQ ID NO: 1. The genome may have an open reading frame 1 (ORF1) nucleotide sequence consisting of 51^{st} to 992^{nd} nucleotides of the nucleotide sequence of SEQ ID NO: 1 and an ORF2 nucleotide sequence complementary to 1037^{th} to 1735^{th} nucleotides of the nucleotide sequence of SEQ ID NO: 1. The ORF1 nucleotide sequence is a positive strand and may encode a replicase protein. The ORF2 nucleotide sequence is a negative strand and may encode a capsid protein. The ORF1 protein and ORF2 protein may respectively have amino acid sequences of SEQ ID NOS: 2 and 3.

PCV2 GC0504 viral particles may be easily obtained since PCV2 GC0504 has high proliferative activity in host cells. Thus, PCV2 GC0504 may be efficiently used to prepare attenuated circovirus vaccines. For example, PCV2 GC0504 may be prepared by cultivating PK-15 cells in 10% fetal bovine serum (FBS) supplemented a Dulbecco's modified Eagle medium (DMEM) (900 ml DMEM, 100 ml of fetal bovine serum (FBS), 100 μg/ml of streptomycin, 200 IU/ml of penicillin, and 100 μg/ml of kanamycin) to form a single layer, removing the medium therefrom, isolating the PK-15 cells therefrom by treatment with trypsin, adding 10^{4.6} TCID₅₀/0.1 ml of PCV2 to a PK-15 cell suspension for inoculation, static-cultivating the cells at 37° C. for 3 to 4 days, completely destroying the cells by freeze-thawing the cells three times, and centrifuging the cells at 2,500 rpm for 15 minutes to obtain a supernatant. In the supernatant, an average content of PCV2 GC0504 is equal to or greater than 10^{5.6} TCID₅₀/ml, for example, in the range of 10^{5.6} to 10^{6.0} TCID₅₀/ml. Thus, it can be seen that PCV2 GC0504 has high proliferative activity. The high proliferative activity of PCV2 GC0504 may also be obtained in the absence of proliferation enhancer, such as glucosamine.

PCV2 GC0505 may include a genome having a nucleotide sequence of SEQ ID NO: 4. The genome may have an ORF1 nucleotide sequence consisting of 51^{st} to 992^{nd} nucleotides of the nucleotide sequence of SEQ ID NO: 4 and an ORF2 nucleotide sequence complementary to 1036^{th} to 1734^{th} nucleotides of the nucleotide sequence of SEQ ID NO: 4. The ORF1 nucleotide sequence is a positive strand and may encode a replicase protein. The ORF2 nucleotide sequence is a negative strand and may encode a capsid protein. The ORF1 protein and ORF2 protein may respectively have amino acid sequences of SEQ ID NOS: 5 and 6.

PCV2 GC0505 viral particles may be easily obtained since PCV2 GC0505 has high proliferative activity in host cells. Thus, PCV2 GC0505 may be efficiently used to prepare attenuated circovirus vaccines. For example, PCV2 GC0505 may be prepared by cultivating PK-15 cells in 10% fetal bovine serum (FBS) supplemented-Dulbecco's modified Eagle medium (DMEM) (900 ml of DMEM, 100 ml of 10% fetal bovine serum (FBS), 100 μg/ml of streptomycin, 200 IU/ml of penicillin, and 100 μg/ml of kanamycin) to form a single layer, removing the medium therefrom, isolating the PK-15 cells therefrom by treatment with trypsin, adding 10^{4.6} TCID₅₀/0.1 ml of PCV2 to a cell suspension for inoculation, static-cultivating the cells at 37° C. for 3 to 4 days, completely destroying the cells by freeze-thawing the cells three times, and centrifuging the cells at 2,500 rpm for 15 minutes to obtain a supernatant. In the supernatant, an average content of PCV2 GC0505 is equal to or greater than 10^{5.6} TCID₅₀/ml, for example, in the range of 10^{5.6} to 10^{6.0} TCID₅₀/ml. Thus, it can be seen that PCV2 GC0505 has high proliferative activity. The high proliferative activity of PCV2 GC0505 may be obtained in the absence of proliferation enhancer, such as glucosamine.

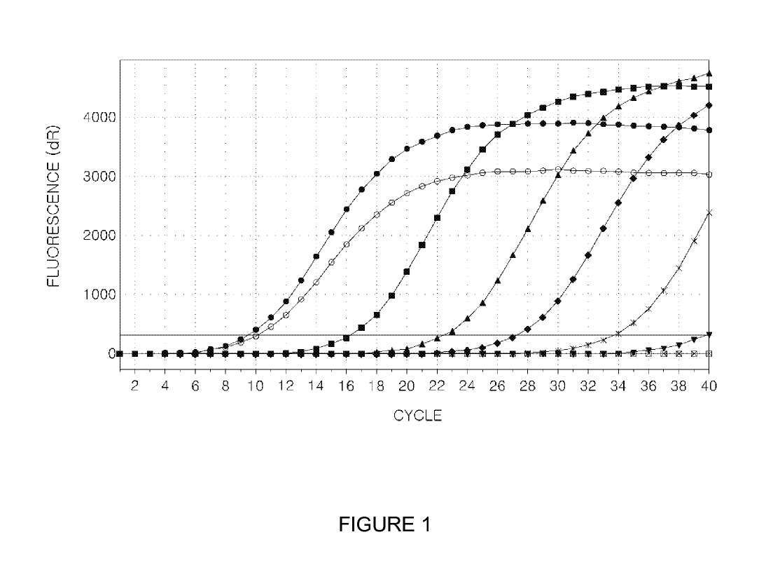

At least one circovirus selected from the group consisting of PCV2 GC0504 and PCV2 GC0505 may be prepared from their genomic sequences by using any known method in the art. The circovirus may be produced by inserting their genomic sequence into a host cell or host animal of PCV2 and isolating infectious PCV2 from the host cell or host animal. The host cell may be a PK-15 cell. The host animal may be a pig. For example, the circovirus may be produced by obtaining genomic DNA of PCV2 GC0504 or PCV2 GC0505 having a nucleotide sequence of SEQ ID NO: 1 or 2, introducing the DNA into a recombinant vector, inserting the recombinant vector into a host cell or host animal of PCV2, and isolating infectious PCV2 from the host cell or host animal. For example, the genomic DNA of PCV2 GC0504 or PCV2 GC0505 having a nucleotide sequence of SEQ ID NO: 1 or 2 may be obtained by DNA synthesis. The whole genomic DNA or fragments of the genomic DNA may be synthesized. When fragments of the genomic DNA are synthesized, the fragments are ligated to prepare the genomic DNA. The ligation may be performed by enzymes such as a DNA ligation enzyme and a DNA kinase. The genomic DNA of PCV2 GC0504 or PCV2 GC0505 with a length of about 1.8 kb may be easily synthesized by those of ordinary skill in the art. The synthesized genomic DNA or a vector including the genomic DNA may be inserted into the host cell or the host animal. The virus may be produced using methods disclosed in U.S. Patent Application Publication No. 200410253270A1 (e.g., Paragraph Nos. 0084 to 0086, Examples 2 to 5, and FIG. 1) and disclosed by Fenaux, M, et. al., (J. Vir. January 2002, p. 541-551), the disclosures of which are incorporated herein in their entireties by reference. If these methods are applied to the production of PCV2 GC0504 or PCV2 GC0505, the whole genome of PCV2 GC0504 or PCV2 GC0505 may be amplified by PCR using the synthesized genomic DNA as a template and a pair of forward and reverse primers having a unique restriction enzyme site, e.g., SacII site. In order to obtain DNA clone including a tandem dimer of PCV2 genome, the PCR products are ligated to a vector, for example, TAge plasmid vector (Clontech, Palo Alto, Clif) to transform E. Coli DHSa competent cell. The vector is isolated from the transformed cell to identify the existence of the whole genome and cut using the restriction enzyme, e.g., SacII, and the cut PCV2 genomic DNA is ligated using a DNA ligase to prepare a tandem dimer. Then, the tandem dimer is cloned in a pBluescript SK(+) vector (pSK) (Stratagene Inc., La Jolla, Calif.) to prepare a recombinant plasmid including a tandem dimer of PCV2 genome (PCV2 molecular DNA clone). Then, the PCV2 molecular DNA clone may be in vitro transfected into PK-15 cells. The transfected PK-15 cells are cultivated and destroyed to isolate the virus to obtain the PCV2. The PCV2 may be inoculated, for example, intranasally inoculated, into a host animal, for example, a pig, in vivo and isolated from the host animal.

The attenuated circoviruses PCV2 GC0504 and PCV2 GC0505 may not infect the host cell and do not cause pathological lesions to have immunogenicity. Alternatively, PCV2 GC0504 and PCV2 GC0505 may infect the host cell but do not cause pathological lesions to have immunogenicity. The pathological lesions may be any symptom occurring in cells, tissues, and individuals caused by the host infection by circovirus known in the art. For example, the pathological lesions may include at least one selected from the group consisting of congenital tremor, postweaning multisystemic wasting syndrome (PMWS), wasting, dyspnea, tachypnea, jaundice, diarrhea, cough, central nervous system instability, weight loss, acute vasculitis (Langohr, 2010, Vet pathol. vol 47, p 140-147), breeding disease (miscarriage, stillbirth, fetal mummification, etc., Hansen, 2010, Vet Microbiol., Epub ahead of print), and porcine dermatitis nepropathy syndrome (PDNS).

The attenuation may be conducted using any method known in the art. For example, the attenuation may be conducted by repeated passages of the circoviruses in cells such as PK-15 cells or by the activation of circovirus-associated endonuclease. In addition, the attenuation may be conducted by chemical methods, for examples, by exposing the virus to formaldehyde (formalin), paraformaldehyde, β-propiolactone, ethyleneimine, or derivatives thereof or by UV-irradiation. For example, at least one virus selected from the group consisting of PCV2 GC0504 and PCV2 GC0505 is subjected to reaction with formaldehyde to be attenuated. In the reaction, the amount of formaldehyde may be in the range of 0.2 to 0.5 wt % based on the total weight of the reaction solution. The reaction may be conducted at room temperature while stirring at 100 rpm for 10 to 24 hours.

The composition may be used to treat or prevent pathological lesions caused by PCV2 infection in animals. For example, the pathological lesions may include at least one selected from the group consisting of congenital tremor, postweaning multisystemic wasting syndrome (PMWS), wasting, dyspnea, tachypnea, jaundice, diarrhea, cough, central nervous system instability, weight loss, and porcine dermatitis nepropathy syndrome (PDNS).

The amount of PCV2 GC0504 and PCV2 GC0505 contained in the composition may vary according to the type of animal, severity of disease, age of the animal, and formulation of the composition. For example, the amount of PCV2 GC0504 and PCV2 GC0505 may be equal to or greater than 10^{4.5} TCID₅₀, for example, in the range of 10^{4.5} TCID₅₀ to 10^{6.5} TCID₅₀ at a single dose.

In the composition, the circovirus to be prevented or treated infections may be selected from the group consisting of PCV2a and PCV2b. For example, the circovirus may include PCV2a and PCV2b. PCV2 is classified into two genotypes, PCV2a and PCV2b, but the PCV2a and PCV2b are not classified into substrains. Thus, the composition including the attenuated PCV2 GC0504 and PCV2 GC0505 according to an embodiment of the present invention may defend viruses belonging to PCV2a and PCV2b, i.e., all PCV2.

The composition may further include a veterinarily acceptable carrier. The carrier may be any carrier commonly used in the art to maintain immunogenicity of the composition. For example, the carrier may include at least one selected from the group consisting of a diluent, an excipient, an adjuvant, and a freeze-drying stabilizer. The diluent may include at least one selected from the group consisting of water such as sterilized water and a buffer solution. The adjuvant may be any adjuvant commonly used in the art to improve immunogenicity of PCV2 GC0504 and PCV2 GC0505. For example, the adjuvant may include at least one selected from the group consisting of aluminium hydroxide, saponin, polyphosphazene, and Montanide Gel™. The freeze-drying stabilizer may be carbohydrates such as sorbitol, mannitol, starch, sucrose, dextran, or glucose, proteins such as albumin or casein, derivatives of these compounds, or buffers such as alkali metal phosphates. The amount of the carrier may vary according to the type of the carrier as well known by those of ordinary skill in the art. The composition may include 5 to 15% of the carrier based on the total weight of the composition.

According to the current embodiment, the composition may include 10^{4.5} TCID₅₀ or greater, e.g., 10^{4.5} TCID₅₀ to 10^{7.5} TCID₅₀, 10^{4.5} TCID₅₀ to 10^{6.5} TCID₅₀, or 10^{6.0} TCID₅₀ to 10^{7.0} TCID₅₀, of PCV2 GC0504 and PCV2 GC0505, respectively per 1 unit dosage of the composition, and 5% to 10% of Montanide Gel™ based on the total weight of the composition. The Montanide Gel™ (SEPPIC, Paris, France) consists of a synthetic polymer and a hydrophilic gel.

The composition may be an immunogenic composition in any formulation known in the art. The composition may be an oral or parenteral formulation. The formulation of the composition may be a freeze-dried formulation, granule, powder, liquid, tablet, capsule, dried syrup, or injection. The freeze-dried formulation may be dissolved in a sterilized liquid carrier, e.g., sterilized water, before use.

The composition may be administered using any method commonly used in the art to administer an immunogenic composition. For example, the composition may be administered into individuals via a route such as an intravenous, intramuscular, oral, transdermal, mucosal, intranasal, intratracheal, or subcutaneous route. The administration may be a systematic administration or a topical administration.

In the composition, the animal may be selected from the group consisting of pig, mouse, and guinea pig.

One or more embodiments of the present invention provide a method of producing an attenuated circovirus, the method including reacting at least one virus selected from the group consisting of PCV2 GC0504 and PCV2 GC0505 with formalin to obtain attenuated circovirus.

The reaction may be conducted by adding the virus into a reaction solution including formalin and maintaining the reaction solution. The amount of formalin may be in the range of 0.2 to 0.5 wt % based on the total weight of the reaction solution. The reaction may be conducted at room temperature. The reaction may be conducted for 10 to 24 hours. The reaction may be conducted while stirring at 100 rpm.

One or more embodiments of the present invention provide a method of treating or preventing circovirus infection in animals, the method including administering the composition having an effective amount into the animals.

The composition may be administered using any method commonly used in the art to administer an immunogenic composition. The composition may be orally or parenterally administered. For example, the composition may be administered into individuals via a route such as an intravenous, intramuscular, oral, transdermal, mucosal, intranasal, intratracheal, rectal, or subcutaneous route. The administration may be a systematic administration or a topical administration. The administration may be conducted by injection or infusion.

The composition may be administered once or more, e.g., twice or more. The composition may be administered twice. For example, the composition may be administered once and administered again after two weeks from the first administration. The animal may be selected from the group consisting of pig, mouse, and guinea pig.

The “effective amount” refers to an amount effective for preventing or treating PCV2 infection in animals. The treatment of prevention includes removing and reducing lesions caused by PCV2 infection in addition to preventing PCV2 infection in animals. The effective amount may vary according to the type of animal, severity of disease, age of the animal, and formulation of the composition. For example, the effective amount of PCV2 GC0504 and PCV2 GC0505 may include 10^{4.5} TCID₅₀ or greater, for example, 10^{4.5} TCID₅₀ to 10^{7.5} TCID₅₀, 10^{4.5} TCID₅₀ to 10^{6.5} TCID₅₀, or 10^{6.0} TCID₅₀ to 10^{7.0} TCID₅₀, of PCV2 GC0504 and PCV2 GC0505, respectively at a single dose.

The composition is defined above. The animal may be any animal in which pathological lesions are caused by PCV2 infection. For example, the animal may be selected from the group consisting of pig, mouse, and guinea pig.

One or more embodiments of the present invention provide circovirus PCV2 GC0504 including a genome having a nucleotide sequence of SEQ ID NO: 1.

The genome may have an ORF1 nucleotide sequence consisting of 51^{st} to 992^{nd} nucleotides of the nucleotide sequence of SEQ ID NO: 1 and an ORF2 nucleotide sequence complementary to 1037^{th} to 1735^{th} nucleotides of the nucleotide sequence of SEQ ID NO: 1. The ORF1 nucleotide sequence is a positive strand and may encode a replicase protein. The ORF2 nucleotide sequence is a negative strand and may encode a capsid protein. The ORF1 protein and ORF2 protein may respectively have amino acid sequences of SEQ ID NOS: 2 and 3.

PCV2 GC0504 may include any viral particles expressed by the genomic sequence of SEQ ID NO: 1. The circovirus PCV2 GC0504 may include at least one amino acid sequence selected from the group consisting of ORF1 and ORF2 having amino acid sequences of SEQ ID NOS: 2 and 3. The circovirus PCV2 GC0504 is a PCV2-2a virus.

Due to high proliferative activity in a host cell, PCV2 GC0504 may be efficiently used to prepare attenuated circovirus vaccine. For example, PCV2 GC0504 may be prepared by cultivating PK-15 cells in 10% fetal bovine serum (FBS) supplemented-Dulbecco's modified Eagle medium (DMEM) (900 ml of DMEM, 100 ml of 10% fetal bovine serum (FBS), 100 μg/ml of streptomycin, 200 IU/ml of penicillin, and 100 μg/ml of kanamycin) to form a single layer, removing the medium therefrom, isolating the PK-15 cells therefrom by treatment with trypsin, adding 10^{4.6} TCID₅₀/0.1 ml of PCV2 to a cell suspension for inoculation, static-cultivating the cells at 37° C. for 3 to 4 days, completely destroying the cells by freeze-thawing three times, and centrifuging the cells at 2,500 rpm for 15 minutes to obtain a supernatant. In the supernatant, an average amount of PCV2 GC0504 is equal to or greater than 10^{5.6} TCID₅₀/ml, for example, in the range of 10^{5.6} to 10^{6.0} TCID₅₀/ml. Thus, it can be seen that PCV2 GC0504 has high proliferative activity. PCV2 GC0504 is a virus belonging to PCV2-2a.

The attenuated circovirus PCV2 GC0504 may have immunogenicity against various species of PCV2a strains.

One or more embodiments of the present invention provide circovirus PCV2 GC0505 including a genome having a nucleotide sequence of SEQ ID NO: 2.

PCV2 GC0505 may include a genome having a nucleotide sequence of SEQ ID NO: 4. The genome may have an ORF1 nucleotide sequence consisting of 51^{st} to 992^{nd} nucleotides of the nucleotide sequence of SEQ ID NO: 4 and an ORF2 nucleotide sequence complementary to 1036^{th} to 1734^{th} nucleotides of the nucleotide sequence of SEQ ID NO: 4. The ORF1 nucleotide sequence is a positive strand and may encode a replicase protein. The ORF2 nucleotide sequence is a negative strand and may encode a capsid protein. The ORF1 protein and ORF2 protein may respectively have amino acid sequences of SEQ ID NOS: 5 and 6. PCV2 GC0505 may include any viral particles expressed by the genomic sequence of SEQ ID NO: 4.

PCV2 GC0505 may include at least one amino acid sequence selected from the group consisting of ORF1 and ORF2 having amino acid sequences of SEQ ID NOS: 5 and 6. PCV2 GC0505 is a virus belonging to PCV2-2b.

Due to high proliferative activity in a host cell, PCV2 GC0505 may be efficiently used to prepare attenuated circovirus vaccine. For example, PCV2 GC0505 may be prepared by cultivating PK-15 cells in 10% fetal bovine serum (FBS) supplemented-Dulbecco's modified Eagle medium (DMEM) (900 ml of DMEM, 100 ml of 8% fetal bovine serum (FBS), 100 μg/ml of streptomycin, 200 IU/ml of penicillin, and 100 μg/ml of kanamycin) to form a single layer, removing the medium therefrom, isolating the PK-15 cells therefrom by treatment with trypsin, adding 10^{4.6} TCID₅₀/0.1 ml of PCV2 to a cell suspension for inoculation, static-cultivating the cells at 37° C. for 3 to 4 days, completely destroying by freeze-thawing three times, and centrifuging the cells at 2,500 rpm for 15 minutes to obtain a supernatant. In the supernatant, an average amount of PCV2 GC0505 is equal to or greater than 10^{5.6} TCID₅₀/ml, for example, in the range of 10^{5.6} to 10^{6.0} TCID₅₀/ml. Thus, it can be seen that PCV2 GC0505 has high proliferative activity.

The attenuated circovirus PCV2 GC0505 may have immunogenicity against various species of PCV2b strains.

Each of PCV2 GC0504 and PCV2 GC0505 may be replicated in vitro or in vivo in a porcine cell line. The cell line may be an immortalized cell line. The cell line may be selected from the group consisting of a porcine kidney epithelial cell lines PK-15 and SK, monomyeloid cell line 3D4/31, and testicular cell line ST.

The nucleotide sequences described herein are attached hereto as a sequence list, the disclosure of which is incorporated herein in its entirety by reference.

The present disclosure will be described in further detail with reference to the following examples. These examples are for illustrative purposes only and are not intended to limit the scope of the invention.

### EXAMPLES

### Example 1

### Isolation of Virus and Identification of Proliferative Activity

PCV was isolated from specimen of pigs requested between 2005 to 2007 using PCR and indirect immunofluorescence assay (IFA). PK-15 cells were infected with the PCV isolate and cultivated to measure the number of proliferated viral particles, and then a PCV isolate having the highest proliferative activity was selected.

First, PK-15 cells were cultivated in 10% fetal bovine serum (FBS) supplemented-Dulbecco's modified Eagle medium (DMEM) (900 ml of DMEM, 100 ml of 10% fetal bovine serum (FBS), 100 μg/ml of streptomycin, 200 IU/ml of penicillin, and 100 μg/ml of kanamycin) at 37° C. to form a single layer. Then, the medium was removed from the PK-15 cells having the single layer, and the PK-15 cells were isolated therefrom by treatment with trypsin. 10^{4.5} TCID₅₀/0.1 ml of PCV2 was added to a virus-containing cell suspension for inoculation, and the cells were static-cultivated at 37° C. for 3 to 4 days. The cultivated cells were freeze-thawed three times to completely destroy the cells and centrifuged the cells at 2,500 rpm for 15 minutes. The amount of PCV2 contained in a supernatant obtained by the centrifugation was measured using PCR and IFA. As a result, it was identified that two PCV2 isolates had high virus contents. Then, the two isolates were referred to as PCV2 GC0504 and PCV2 GC0505. PCV2 GC0504 is a PCV2-2a virus and has a genomic sequence of SEQ ID NO: 1. PCV2 GC0505 is a PCV2-2b virus and has a genomic sequence of SEQ ID NO: 2. PCV2 GC0504 and PCV2 GC0505 are replicated such that an average amount thereof is in the range of 10^{5.6} to 10^{7.0} TCID₅₀/ml in a culture medium. It was identified that PCV2 GC0504 and PCV2 GC0505 have more than 10 times higher proliferative activity than conventional PCV2. Thus, at least one virus selected from the group consisting of PCV2 GC0504 and PCV2 GC0505 may efficiently provide viruses required for preparing attenuated or inactivated vaccine. Thus, the obtained PCV2 (bulk virus) was used to attenuate the following viruses.

In this regard, the PCR was real time-PCR. In the real time-PCR, a SYBR green manufactured by Takara was used as a reagent. A sample obtained by diluting 10^{6.0} TCID₅₀/ml of PCV2 GC0505 by ten times was used as a template and primers specific to ORF2 of PCV2 (5′-TAG GTT AGG GCT GTG GCC TT-3′ forward primer, SEQ ID NO: 7 and 5′-CCG CAC CTT CGG ATA TAO TG-3′ reverse primer, SEQ ID NO: 8) were used. The sample was maintained at 94° C. for 10 seconds, at 94° C. for 5 seconds, and at 60° C. for 40 seconds, and this process was repeated 40 times. The amplified product was anticipated to be 263 bp. As a result, the virus was successfully detected at a concentration of 10 TCID₅₀/ml (FIG. 1). FIG. 1 is a graph illustrating results of real time-polymerase chain reaction (PCR) of PCV2 having various concentrations.

The relationship between Ct value measured by the real time-PCR and virus titer measured by IFA is defined as in Formula 1 below.

y=4.1291x+4.458 Formula 1

In Formula 1, y is Ct value, x is IFA titer (log₁₀), and the unit is TCID₅₀/0.1 ml. According to Formula 1, the correlation coefficient (R²) is 0.9988, indicating about 99.9% reliability. Since the real time-PCR showed high sensitivity capable of detecting the virus concentration of 10 TCID₅₀/1 ml and was quickly performed, it was efficiently used to measure the virus.

PCV2-specific antibody was measured using sandwich indirect enzyme-linked immunosorbent assay (ELISA).

First, the PCV2-specific antibody was diluted in a coating buffer including 2.93 g of NaHCO₃, 1.59 g of Na₂CO₃, 1000 ml of water, and 0.5 M carbonate buffer (pH 9.6) to a concentration of 5 to 10 μg/well, and 100 μl of the diluted solution was added to each well of an ELISA plate and adsorbed thereto at 37° C. for 2 hours. The ELISA plate to which the antibody was adsorbed was washed three times with PBS-T. 200 μl of a blocking buffer (BSA 1-2 g, PBS 100 ml: 1-2% BSA) was added to each well and the plate was maintained at 37° C. for 2 hours. The plate was washed three times. Then, the PCV2 antigen was diluted 10 times to 20 times with PBS, and the diluted solution was added to each well and the plate was maintained at 37° C. for 1 hour. The plate was washed three times. Then a diluted sample (serum) obtained by diluting the sample (serum) with a dilution buffer including 8.0 g of NaCl, 0.2 g of anhydrous KH₂PO₄, 1.19 g of anhydrous Na₂HPO₄, 0.2 g of KCl, and 1000 ml of water (pH 7.2-7.4) to a ratio of 1:50 was added to each well of the plate in which the antigen was sensitized, and the plate was maintained at 37° C. for 1 hour. The plate was washed five times with a washing buffer (Tween 20 0.5 ml, PBS 1,000 ml: 0.05% PBST), and 100 μl of anti-GP IgG HRP conjugate diluted with the dilution buffer was added thereto. The mixture was maintained at 37° C. for 1 hour. The plate was washed five times, and 100 μl of OPD substrate was added to each well of the plate. The plate was maintained at room temperature for 10 minutes, and 50 μl of a stop solution (2.5M H₂SO₄) was added thereto, and then absorbance was measured at 492 nm.

The isolated PCV2 GC0504 and PCV2 GC0505 were inoculated into PK-15 cells having a single layer, and the cells were cultivated at 37° C. for 72 hours to harvest a virus-infected solution. The solution was centrifuged at 2,500 rpm for 15 minutes, and the supernatant or freeze-dried supernatant was preserved at a temperature equal to or less than −80° C.

### Example 2

### Attenuation of Virus and Preparation of Immunogenic Composition Including Attenuated Virus

(2.1) Attenuation of Virus

Among the bulk virus harvested Operation 1 above, bulk virus having a concentration of 10^{6.0} TCID₅₀/ml or greater was used for the attenuation. Formalin was added in a concentration of 0.3% to the bulk virus in 10% FBS supplemented-DMEM (900 ml of a DMEM, 100 ml of 10% FBS, 100 μg/ml of streptomycin, 200 IU/ml of penicillin, and 100 μg/ml of kanamycin), and the medium was maintained at room temperature for 24 hours to attenuate PCV2 GC0504 and PCV2 GC0505. The attenuated PCV2 GC0504 and PCV2 GC0505 were used as a bulk material to prepare an immunogenic composition.

(2.2) Preparation of Immunogenic Composition

The concentration of each of PCV2 GC0504 and PCV2 GC0505 prepared in Operation (2.1) was respectively set to 10^{6.0} TCID₅₀/ml, and PCV2 GC0504 and PCV2 GC0505 are mixed at a ratio of 45:45. Motanide Gel™ was added thereto to a concentration of 10% (w/w) and the mixture was stirred at room temperature at 100 rpm for more than 1 hour. The pH of the prepared immunogenic composition was maintained in the range of 6.0-8.0, and the concentration of formalin was maintained to be equal to or less than 0.3 wt %. The immunogenic composition prepared herein will be used in the following processes unless otherwise stated. Hereinafter, the immunogenic composition is also referred to as a “vaccine”.

### Example 3

### Identification of Attenuation and Stability

(3.1) Identification of Attenuation

The attenuated sample was centrifuged and an appropriate amount of the sample was added to a dialysis membrane. The sample was dialyzed overnight in a phosphate buffer solution (PBS) in an amount 100 times or greater than the sample at 4° C. to remove the attenuating agent. The resultant was used for experiments.

Primary porcine kidney cells or PK-15 cells were proliferated in a flask (25 cm²) for tissue cultivation. The primary cells were cells cultured for 4 to 7 days, and it was identified that the PK-15 cells were not infected with PCV1 before experiments. The medium was removed from the proliferated cells, and 1 ml of the sample was inoculated into the tissue-cultivated cells and sensitized at 37° C. for 1 hour. Then, a new culture medium for maintaining the cells was added thereto, and the cells were further cultivated for 7 days to identify cytopathic effect (CPE). When the supernatant was removed, the proliferation of the porcine circovirus was identified using IFA.

As a result, CPE caused by the porcine circovirus or any other virus was not observed in the primary porcine kidney cells or PK-15 cells. In addition, in the supernatant, hemagglutination (HA) against chickens and pigs blood cells was not observed, and no virus was detected by IFA.

(3.2) Identification of Stability

Eight mice having weights in the range of 15 to 20 g and 4 guinea pigs having weights in the range of 300 to 400 g were used. In addition, 2 healthy young pigs between 3 to 5 weeks of age which were not inoculated with PCV2-associated vaccine and not exposed to PCV2 were used.

0.5 ml (10^{6.0} TCID₅₀/ml) of the composition was intraperitoneally inoculated into the 8 mice, 2 ml (10^{6.0} TCID₅₀/ml) of the composition was intramuscularly or subcutaneously inoculated into the 4 guinea pigs, and 2 ml of the composition was intraperitoneally inoculated into the two young pigs, and the animals were observed for 7 days whether they were alive. After a single dose of 10^{6.0} TCID₅₀/ml was inoculated into muscle on the right of the neck of the 2 young pigs, the 2 young pigs were observed whether the pigs had hypersensitive reactions or fever within 1 to 2 hours and whether the pigs survived without side effects such as purulence or necrosis for 21 days. As a result of inoculating the composition with varying lots into the mice, guinea pigs, and young pigs, they were alive without any side effect during the experiments.

(3.3) Antibody Titer

The immunogenic composition was administered into animals, and antibody titers of serums were measured. The antibody titer was measured using sandwich indirect ELISA or IFA.

Ten guinea pigs having weights in the range of 300 to 350 g were prepared. Eight of them were used as an inoculation group, and 2 of them were used as a control group. 0.5 ml (10^{6.0} TCID₅₀/ml) of the composition was subcutaneously inoculated into the inoculation group. After 3 weeks, blood samples of the 10 guinea pigs were taken, and antibody titers thereof were measured using IFA or sandwich indirect ELISA.

The antibody titer measured using sandwich indirect ELISA was calculated by dividing an average absorbance (O.D.) of the inoculation group T by an average absorbance (O.D.) of the control group C. When the T/C was 2.0 or higher, the T/C for an average antibody titer of the inoculation group obtained by IFA was 128 times or greater, and the control group was negative, it was determined that the immunogenic composition was appropriate. The antibody titers measured using ELISA are shown in Table 1 below.

TABLE 1

| No. | Group | Absorbance | Absorbance (T/C) |
|---|---|---|---|
| 1 | Inoculation | 0.88 | 2.9 |
| | Control | 0.30 | |
| 2 | Inoculation | 0.82 | 3.4 |
| | Control | 0.24 | |
| 3 | Inoculation | 0.87 | 3.0 |
| | Control | 0.29 | |
| 4 | Inoculation | 0.88 | 2.9 |
| | Control | 0.30 | |

As shown in Table 1, the immunogenic composition has immunogenicity suitable for the inoculation group (experimental group) compared to the control group.

### Example 4

### Identification of Effectiveness of Immunogenic Composition

Here, a minimum amount of the immunogenic composition required for inducing immunity in animals (amount of antigen required for minimum immunity) is determined, and effectiveness such as immunity-inducing ability and weight gain rate when the immunogenic composition is inoculated into animals is identified.

The same immunogenic composition was used except that PCV2 titers of 10^{3.5}, 10^{4.5}, 10^{5.5}, and 10^{6.5} TCID₅₀/ml were used to measure the amount of antigen required for minimum immunity (4 times repeated). The immunogenic composition was used to measure the effectiveness.

Experiments to determine the amount of antigen required for minimum immunity were performed using young pigs. PCV2 vaccines having antigen contents of 10^{3.5}, 10^{4.5}, 10^{5.5}, and 10^{6.5} TCID₅₀/ml were inoculated twice into fifteen 3-week old young pigs which are negative against porcine circoviruses at 2-week intervals, and three young pigs were used as a control group. Blood samples of the young pigs were taken before the inoculation and after two weeks from the second inoculation to measure antibody titers of PCV2.

The effectiveness of the immunogenic composition was tested using young pigs. The immunogenic composition was inoculated twice into four 3-week old young pigs which were negative against porcine circovirus at 2-week intervals, and two young pigs were used as a control group. Blood samples of the young pigs were taken after 2 weeks, 4 weeks, 2 months, 3 months, and 4 months after the second inoculation, and PCV antibody titer was measured using ELISA and serum neutralizing (SN) antibody titer using IFA were measured as described above (Fort, et al., 2007:Vet Microbiol 125, p 244-255). The antibody titer obtained using ELISA indicates all antibodies induced by the antigen, and the SN antibody titer indicates a serum neutralizing antibody titer that is an antibody level capable of neutralizing pathogens such as virus among the antibodies existing in the serum.

In addition, the effect of the immunogenic composition to a weight gain rate of the young pigs was measured. Weights of the young pigs were measured before the first inoculation, before the second inoculation, after two weeks from the second inoculation, and after six weeks from the second inoculation to identify the weight gain rate.

As results of measuring the amount of the antigen required for the minimum immunity and the antibody titer after the second inoculation using ELISA, the change of the antibody titer was identified at 10^{3.5} TCID₅₀/ml. However, when the antigen content was greater than 10^{4.5} TCID₅₀/ml, the antibody titer was significantly increased (p<0.05) when compared to the control group (Table 2).

TABLE 2
Immunogenicity according to PCV2 content (ELISA)

| | Antibody | | | | | |
|---|---|---|---|---|---|---|
| | titer & | PCV2 content (TCID₅₀/ml) | | | | Control |
| Time | significance | 10^{3.5} | 10^{4.5} | 10^{5.5} | 10^{6.5} | — |
| Before inoculation | M* | 0.59 | 0.65 | 0.45 | 0.57 | 0.47 |
| | Significance | 0.24 | 0.46 | 0.34 | 0.39 | |
| After 2 weeks | M* | 0.49 | 0.69 | 0.73 | 0.71 | 0.37 |
| from 2^{nd} | Significance | 0.18 | <0.05 | <0.05 | <0.05 | |
| inoculation | | | | | | |

M* is a geometric mean antibody titer.

Table 3 shows antibody titers of young pigs into which the immunogenic composition was inoculated.

TABLE 3
Antibody titers of young pigs against PCV2 after
the inoculation of the immunogenic composition.

| | | PCV2 | | | |
|---|---|---|---|---|---|
| | | V^{a} | | NV^{b} | |
| | Antibody titer | ELISA | SN | ELISA | SN |
| Time | & significance | Value | titer | Value | titer |
| Before 1^{st} | M^{c} | 0.31 | <2 | 0.35 | <2 |
| inoculation | Significance | 0.34 | | | |
| Before 2^{nd} | M^{c} | 0.47 | <2 | 0.36 | <2 |
| inoculation | Significance | 0.19 | | | |
| After 2 weeks from | M^{c} | 0.77 | 48 | 0.32 | <2 |
| 2^{nd} inoculation | Significance | <0.05 | <0.05 | | |
| After 4 weeks from | M^{c} | 0.84 | 68 | 0.37 | <2 |
| 2^{nd} inoculation | Significance | <0.05 | <0.05 | | |
| After 2 months from | M^{c} | 0.81 | 64 | 0.35 | <2 |
| 2^{nd} inoculation | Significance | <0.05 | <0.05 | | |
| After 3 months from | M^{c} | 0.78 | 62 | 0.37 | <2 |
| 2^{nd} inoculation | Significance | <0.05 | <0.05 | | |
| After 4 months from | M^{c} | 0.80 | 62 | 0.35 | <2 |
| 2^{nd} inoculation | Significance | <0.05 | <0.05 | | |

M* is a mean value.
^{a}V (vaccinated group)(n = 4)
^{b}NV (non-vaccinated group)(n = 2)

Tables 4 and 5 show weights of young pigs inoculated with the immunogenic composition. Tables 4 and 5 show weights of the inoculation group and control group before the first inoculation, before the second inoculation, after two weeks from the second inoculation, and after 6 weeks from the second inoculation. An average weight of the inoculation group after two weeks from the second inoculation, i.e., seven-week old, was greater than that of the control group by about 1 kg, and an average weight of the inoculation group at the age of 13 weeks was greater than that of the control group by about 4 kg.

TABLE 4
weights of the inoculation group (kg)

| | | | After 2 weeks | After 6 weeks |
|---|---|---|---|---|
| | Before | Before 2^{nd} | from 2^{nd} | from 2^{nd} |
| Individual | inoculation, | inoculation, | inoculation, | inoculation, |
| No. | 3-week old | 5-week old | 7-week old | 13-week old |
| 1 | 5.5 | 8.3 | 15.6 | 41.3 |
| 2 | 4.6 | 7.9 | 15.7 | 38.2 |
| 3 | 4.8 | 8.1 | 14.9 | 39.5 |
| 4 | 6.1 | 9.2 | 16.2 | 45.2 |
| 5 | 4.9 | 8.5 | 15.6 | 47.9 |
| 6 | 5.3 | 8.8 | 15.3 | 38.3 |
| 7 | 4.5 | 7.6 | 16.1 | 35.2 |
| 8 | 4.8 | 8.3 | 15.9 | 40.6 |
| 9 | 5.5 | 8.7 | 14.5 | 41.2 |
| 10 | 5.3 | 7.1 | 16.9 | 43.5 |
| Average | 5.13 | 8.25 | 15.67 | 41.09 |

TABLE 5
weights of the control group (kg)

| Individual No. | 3-week old | 5-week old | 7-week old | 13-week old |
|---|---|---|---|---|
| 1 | 5.5 | 8.5 | 14.5 | 37.5 |
| 2 | 4.8 | 8.8 | 14.3 | 37.3 |
| 3 | 5.1 | 8.2 | 14.7 | 38.2 |
| 4 | 4.7 | 7.9 | 14.8 | 39.1 |
| 5 | 5.2 | 8.3 | 15.2 | 34.2 |
| 6 | 5.1 | 8.9 | 14.1 | 38.1 |
| 7 | 4.3 | 7.6 | 14.6 | 37.4 |
| 8 | 5.3 | 7.3 | 14.8 | 38.2 |
| 9 | 5.1 | 7.8 | 14.6 | 39.1 |
| 10 | 4.9 | 7.4 | 15.2 | 35.9 |
| Average | 5.00 | 8.07 | 14.68 | 37.50 |

In order to identify the effectiveness of the immunogenic composition including attenuated porcine circovirus, antibody titers were measured after the immunogenic composition was inoculated into animals. As a result, in the inoculation group, an increase in the antibody titer was greater than that in the control group. When the immunogenic composition having various concentrations was inoculated into the young pigs, antibody titers significantly increased at a concentration equal to or greater than 10^{4.5} TCID₅₀/ml. Thus, the concentration of the attenuated virus contained in the immunogenic composition needs to be at least 10^{4.5} TCID₅₀/ml. According to the immunogenicity test, the inoculated young pigs showed far greater antibody titers than those of the control group. According to the weight gain rate test, the young pigs of the inoculation group showed greater weight gain rates than those the control group.

### Example 5

### Challenge-Inoculation Test of Young Pig

The immunogenic composition was inoculated into young pigs, and PCV2 was inoculated into the pigs to identify whether defense capability of the pigs was improved by the immunogenic composition.

The immunogenic composition was prepared according to Operation (2.2) above. Fourteen 3-week old young pigs were used for the challenge-inoculation. PCV2 strain isolated from a veterinary medicine (Green Cross Corp.) was used as a virus strain for challenge-inoculation of the young pig. PCV2 was cultivated in PK-15 cells at 37° C. for 48 hours and centrifuged at 500 rpm for 10 minutes. The supernatant was diluted to a concentration of 10^{5.0} TCID50/ml.

Among the fourteen 3-week old specific pathogen free (SPF) young pigs used to identify their defense capability, 1 ml (10^{6.0} TCID₅₀/ml) of the immunogenic composition was intramuscularly inoculated into 8 young pigs twice at 2-week intervals (experimental group), and the other 6 young pigs were not vaccinated (control group).

After two weeks from the second inoculation, the experimental group and the control group were respectively classified into two sub-groups, and challenge-inoculation was conducted with the prepared virus strain. Then, clinical symptoms were observed for 14 days.
First experimental group (PCV2 challenge-inoculation): In the first experimental group, 1 ml of the immunogenic composition was intramuscularly inoculated into four 3-week old young pigs twice at 2-week intervals, and PCV2 was challenge-inoculated after two weeks therefrom (at the age of 7 weeks).
First control group (PCV2 challenge-inoculation): In the first control group, two 3-week old young pigs were raised, and PCV2 was challenge-inoculated into the pigs at the age of 7 weeks.
Second experimental group (PCV2+PRRSV challenge-inoculation): In the second experimental group, 1 ml of the immunogenic composition was intramuscularly inoculated into four 3-week old young pigs twice at 2-week intervals, and PCV2 and PRRSV was challenge-inoculated after two weeks therefrom (at the age of 7 weeks).
Second control group (PCV2+PRRSV challenge-inoculation): In the second control group, two 3-week old young pigs were raised, and PCV2 and PRRSV were challenge-inoculated into the pigs at the age of 7 weeks.
No-inoculation control group: In the no-inoculation control group, two 3-week old young pigs were not vaccinated and not inoculated at seven-week old.

In addition, it was identified whether PCV2 was eluted from the challenge-inoculated young pigs. The elution of the virus was identified by swabbing nasal cavity and rectum with sterilized cotton sticks at the day of challenge-inoculation and after 3 days, 7 days, 10 days, 14 days, 17 days, 20 days, 25 days, and 31 days from the challenge-inoculation and measuring the virus content. The PCV2 content was measured using real time-PCR (SYBR Premix Ex Taq 2X, TAKARA™). DNA extracted from 100 μl of PBS-derived samples obtained from the nasal cavity and rectum was used as a template and real time-PCR was conducted using PCR primers described below and under the following conditions. An IFA titer was obtained using the Ct value obtained using the real time-PCR based on the relationship between the Ct value and the IFA, i.e., using Formula 1.

The real-time PCR was conducted by using primers specific to ORF2 of PCV2 (5′-TAG GTT AGG GCT GTG GCC TT-3′ forward primer, SEQ ID NO: 7, and 5′-CCG CAC CTT CGG ATA TAO TG-3′ reverse primer, SEQ ID NO: 8) at 95° C. for 10 seconds, at 95° C. for 5 seconds, and at 60° C. for 40 seconds, and this process was repeated 30 time, and then the resultant was maintained at 95° C. for 1 minutes, at 55° C. for 30 seconds, and at 95° C. for 30 seconds. The amplified product was anticipated to be 263 bp. The mixture for the real time-PCR had a total volume of 25 μl and included 9.5 μl of distilled water, 12.5 μl of SYBR Premix Ex Taq 2X, 0.5 μl of the forward primer (10 pmol), 0.5 μl of the reverse primer (10 pmol), and 2 μl of the template (DNA).

As a result of the challenge-inoculation, there were no particular symptoms in the first experimental group into which PCV2 was challenge-inoculated and the second experimental group into which PCV2 and PRRSV were challenge-inoculated. However, porcine circovirus associated disease (PCVAD) such as eye mucus and diarrhea were observed in the control group into which the immunogenic composition was not inoculated but the PCV2 was inoculated. In particular, typical PCV2-associated symptoms such as depression, watery diarrhea, rough hair, atrophy, and enlarged lymph nodes were observed in the first control group into which PCV2 was challenge-inoculated and in the second control group into which PCV2 and PRRSV were simultaneously challenge-inoculated after one week from the challenge-inoculation. In the second control group, one of the young pig was dead at the 11^{th} day from the challenge-inoculation. The experimental group into which the immunogenic composition was inoculated and the no-inoculation control group exhibited normal health conditions.

Table 6 shows clinical symptoms of the first experimental group into which PCV2 was challenge-inoculated after the challenge-inoculation.

TABLE 6

| | | No. of individual having clinical symptoms | | | | | | | | | | | | | |
|---|---|---|---|---|---|---|---|---|---|---|---|---|---|---|---|
| | DPI | 1 | 2 | 3 | 4 | 5 | 6 | 7 | 8 | 9 | 10 | 11 | 12 | 13 | 14 |
| Fever | Normal | 3 | 4 | 4 | 4 | 4 | 4 | 4 | 4 | 4 | 4 | 4 | 4 | 4 | 4 |
| | Depressed | 1 | 0 | 0 | 0 | 0 | 0 | 0 | 0 | 0 | 0 | 0 | 0 | 0 | 0 |
| | High fever | 0 | 0 | 0 | 0 | 0 | 0 | 0 | 0 | 0 | 0 | 0 | 0 | 0 | 0 |
| Breathing | Normal | 4 | 4 | 4 | 4 | 4 | 4 | 4 | 4 | 4 | 4 | 4 | 4 | 4 | 4 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 4 | 4 | 4 | 4 | 4 | 4 | 4 | 4 | 4 | 4 | 4 | 4 | 4 | 4 |
| | Intermittence | 0 | 0 | 0 | 0 | 0 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 4 | 4 | 4 | 4 | 4 | 4 | 4 | 4 | 4 | 4 | 4 | 4 | 4 | 4 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 4 | 4 | 4 | 4 | 4 | 4 | 4 | 4 | 4 | 4 | 4 | 4 | 4 | 4 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 4 | 4 | 4 | 4 | 4 | 4 | 4 | 4 | 4 | 4 | 4 | 4 | 4 | 4 |
| | Watery diarrhea | 0 | 0 | 0 | 0 | 0 | 0 | 0 | 0 | 0 | 0 | 0 | 0 | 0 | 0 |
| | Diarrhea | 0 | 0 | 0 | 0 | 0 | 0 | 0 | 0 | 0 | 0 | 0 | 0 | 0 | 0 |

Table 7 shows clinical symptoms of the first control group into which PCV2 was challenge-inoculated (non-vaccinated group) after the challenge-inoculation.

TABLE 7

| | | No. of individual having clinical symptoms | | | | | | | | | | | | | |
|---|---|---|---|---|---|---|---|---|---|---|---|---|---|---|---|
| | DPI | 1 | 2 | 3 | 4 | 5 | 6 | 7 | 8 | 9 | 10 | 11 | 12 | 13 | 14 |
| Fever | Normal | 1 | 1 | 1 | 1 | 2 | 2 | 2 | 2 | 2 | 2 | 2 | 2 | 2 | 2 |
| | Depressed | 1 | 1 | 1 | 1 | 0 | 0 | 0 | 0 | 0 | 0 | 0 | 0 | 0 | 0 |
| | High fever | 0 | 0 | 0 | 0 | 0 | 0 | 0 | 0 | 0 | 0 | 0 | 0 | 0 | 0 |
| Breathing | Normal | 2 | 2 | 2 | 2 | 2 | 2 | 2 | 2 | 2 | 2 | 2 | 2 | 2 | 2 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 2 | 2 | 1 | 2 | 1 | 2 | 1 | 2 | 1 | 2 | 2 | 2 | 2 | 1 |
| | Intermittence | 0 | 0 | 1 | 0 | 1 | 0 | 1 | 0 | 1 | 0 | 0 | 0 | 0 | 1 |
| | Severe | 0 | 0 | 0 | 0 | 0 | 0 | 0 | 0 | 0 | 0 | 0 | 0 | 0 | 0 |
| Skin rashes | Normal | 2 | 2 | 2 | 2 | 2 | 2 | 2 | 2 | 2 | 2 | 2 | 2 | 2 | 2 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 2 | 2 | 2 | 2 | 2 | 2 | 2 | 2 | 2 | 2 | 2 | 2 | 2 | 2 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 1 | 1 | 2 | 2 | 2 | 1 | 1 | 1 | 1 | 1 | 1 | 2 | 2 | 2 |
| | Watery diarrhea | 1 | 1 | 0 | 0 | 0 | 1 | 1 | 1 | 1 | 1 | 1 | 0 | 0 | 0 |
| | Diarrhea | 0 | 0 | 0 | 0 | 0 | 0 | 0 | 0 | 0 | 0 | 0 | 0 | 0 | 0 |

Table 8 shows clinical symptoms of the second experimental group into which PCV2 and PRRSV were challenge-inoculated (vaccinated group) after the challenge-inoculation.

TABLE 8

| | | No. of individual having clinical symptoms | | | | | | | | | | | | | |
|---|---|---|---|---|---|---|---|---|---|---|---|---|---|---|---|
| | DPI | 1 | 2 | 3 | 4 | 5 | 6 | 7 | 8 | 9 | 10 | 11 | 12 | 13 | 14 |
| Fever | Normal | 1 | 1 | 3 | 1 | 3 | 3 | 2 | 4 | 3 | 4 | 3 | 4 | 4 | 4 |
| | Depressed | 2 | 2 | 0 | 1 | 1 | 0 | 1 | 0 | 0 | 0 | 0 | 0 | 0 | 0 |
| | High fever | 1 | 1 | 1 | 2 | 0 | 1 | 1 | 0 | 1 | 0 | 1 | 0 | 0 | 0 |
| Breathing | Normal | 3 | 3 | 3 | 4 | 2 | 3 | 3 | 3 | 4 | 4 | 4 | 4 | 4 | 4 |
| | Increase | 1 | 1 | 1 | 0 | 2 | 1 | 1 | 1 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 4 | 4 | 4 | 4 | 4 | 4 | 4 | 4 | 4 | 4 | 4 | 4 | 4 | 4 |
| | Intermittence | 0 | 0 | 0 | 0 | 0 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 4 | 4 | 4 | 4 | 4 | 4 | 4 | 4 | 4 | 4 | 4 | 4 | 4 | 4 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 4 | 4 | 4 | 4 | 4 | 4 | 4 | 4 | 4 | 4 | 4 | 4 | 4 | 4 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 3 | 2 | 4 | 3 | 3 | 4 | 3 | 4 | 4 | 3 | 4 | 3 | 3 | 4 |
| | Watery diarrhea | 1 | 2 | 0 | 1 | 1 | 0 | 1 | 0 | 0 | 1 | 0 | 1 | 1 | 0 |
| | Diarrhea | 0 | 0 | 0 | 0 | 0 | 0 | 0 | 0 | 0 | 0 | 0 | 0 | 0 | 0 |

Table 9 shows clinical symptoms of the second control group into which PCV2 and PRRSV were challenge-inoculated (non-vaccinated group) after the challenge-inoculation.

TABLE 9

| | | No. of individual having clinical symptoms | | | | | | | | | | | | | |
|---|---|---|---|---|---|---|---|---|---|---|---|---|---|---|---|
| | DPI | 1 | 2 | 3 | 4 | 5 | 6 | 7 | 8 | 9 | 10 | 11 | 12 | 13 | 14 |
| Fever | Normal | 1 | 1 | 1 | 1 | 2 | 2 | 2 | 2 | 2 | 2 | 2 | 1 | 1 | 1 |
| | Depressed | 1 | 0 | 1 | 1 | 0 | 0 | 0 | 0 | 0 | 0 | 0 | 0 | 0 | 0 |
| | High fever | 0 | 0 | 0 | 0 | 0 | 0 | 0 | 0 | 0 | 0 | 0 | 0 | 0 | 0 |
| Breathing | Normal | 2 | 2 | 2 | 2 | 2 | 1 | 1 | 0 | 0 | 0 | 0 | 0 | 0 | 0 |
| | Increase | 0 | 0 | 0 | 0 | 0 | 1 | 1 | 1 | 1 | 1 | 1 | 0 | 0 | 0 |
| | Opening mouth | 0 | 0 | 0 | 0 | 0 | 0 | 0 | 1 | 1 | 1 | 1 | 1 | 1 | 1 |
| Cough | Normal | 2 | 2 | 2 | 2 | 2 | 2 | 1 | 1 | 0 | 0 | 0 | 0 | 0 | |
| | Intermittence | 0 | 0 | 0 | 0 | 0 | 0 | 1 | 1 | 2 | 2 | 1 | 1 | 1 | 1 |
| | Severe | 0 | 0 | 0 | 0 | 0 | 0 | 0 | 0 | 0 | 0 | 1 | 0 | 0 | 0 |
| Skin rashes | Normal | 2 | 2 | 2 | 2 | 2 | 2 | 2 | 2 | 2 | 2 | 2 | 1 | 1 | 1 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 2 | 2 | 2 | 2 | 2 | 2 | 1 | 1 | 1 | 1 | 1 | 0 | 1 | 1 |
| | Abnormal | 0 | 0 | 0 | 0 | 0 | 0 | 1 | 1 | 1 | 1 | 1 | 1 | 0 | 0 |
| | Daudication | 0 | 0 | 0 | 0 | 0 | 0 | 0 | 0 | 0 | 0 | 0 | 0 | 0 | 0 |
| Diarrhea | Normal | 2 | 2 | 2 | 2 | 2 | 2 | 1 | 1 | 0 | 0 | 0 | 0 | 0 | 0 |
| | Watery diarrhea | 0 | 0 | 0 | 0 | 0 | 0 | 1 | 1 | 1 | 1 | 1 | 0 | 0 | 0 |
| | Diarrhea | 0 | 0 | 0 | 0 | 0 | 0 | 0 | 0 | 1 | 1 | 1 | 1 | 1 | 1 |

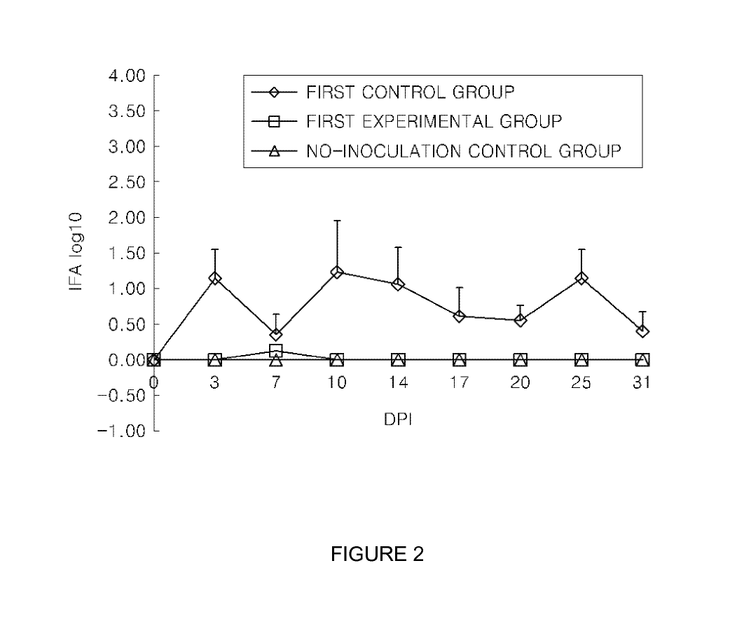

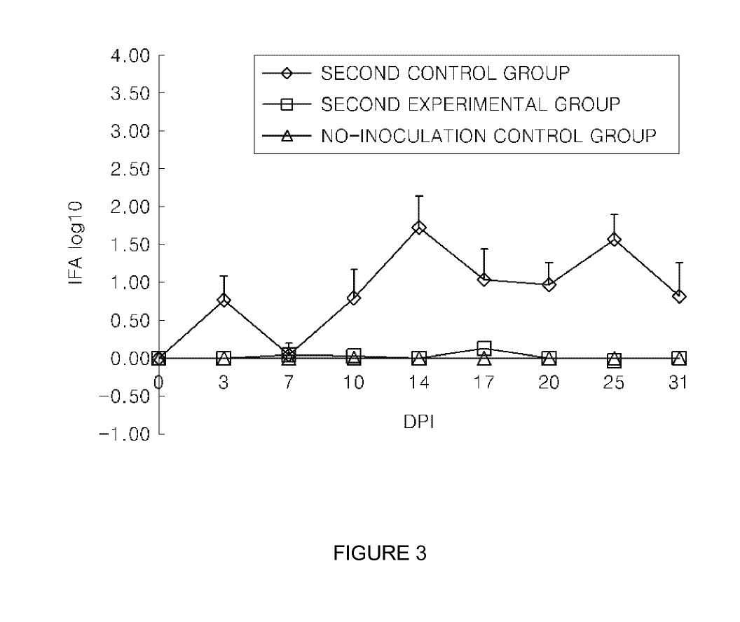

As a result of measuring the elution of the virus via the nasal cavity, it was identified that the PCV2 elution via the nasal cavity was significantly reduced in the first experimental group into which the vaccine was inoculated and the PCV2 was challenge-inoculated and in the second experimental group into which the vaccine was inoculated and PCV2 and PRRSV were challenge-inoculated when compared with the control groups. While the maximum amount of the eluted PCV2 was 10^{0.12} TCID₅₀/0.1 μl per day in the first and second experimental groups, the maximum amount of the eluted PCV2 was 10^{0.81} TCID₅₀/0.1 μl in the control groups. In the first control group inoculated with PCV2 and the second control group inoculated with PCV2 and PRRSV, PCV2 was detected since the 3^{rd} day of inoculation. While the maximum amount of the eluted PCV2 was 10^{1.17} TCID₅₀/0.1 μl per day in the first control group, the maximum amount of the eluted PCV2 was 10^{1.73} TCID₅₀/0.1 μl in the second control group. In the no-inoculation control group, PCV2 elution was not identified (FIGS. 2 and 3).

FIG. 2 is a graph illustrating PCV2 elution via nasal cavity after PCV2 challenge-inoculation into young pigs. Referring to FIG. 2, DPI indicates date after post infection, IFA log₁₀ of the y axis indicates IFA titer obtained using Ct values measured using real time-PCR.

FIG. 3 is a graph illustrating PCV2 elution via nasal cavity after challenge-inoculation of PCV2 and porcine reproductive and respiratory syndrome virus (PRRSV) into young pigs. In FIG. 3, the x and y axes are defined with reference to FIG. 2.

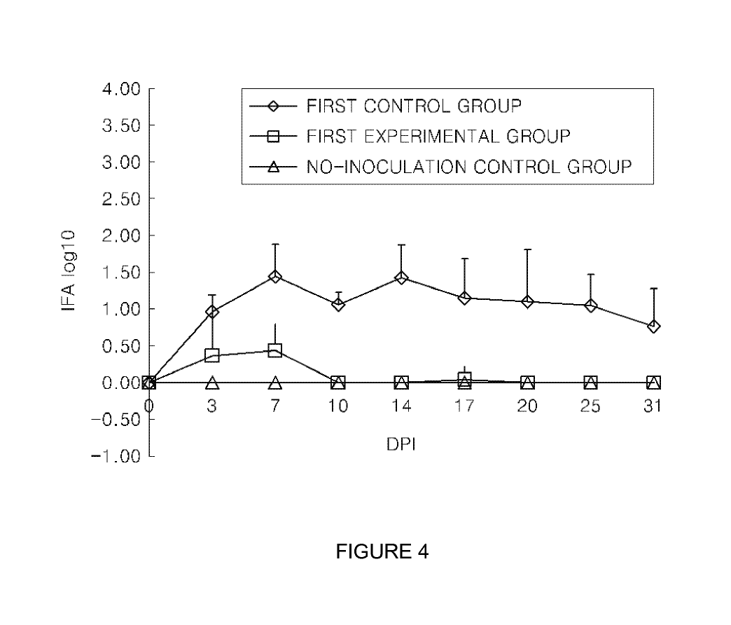

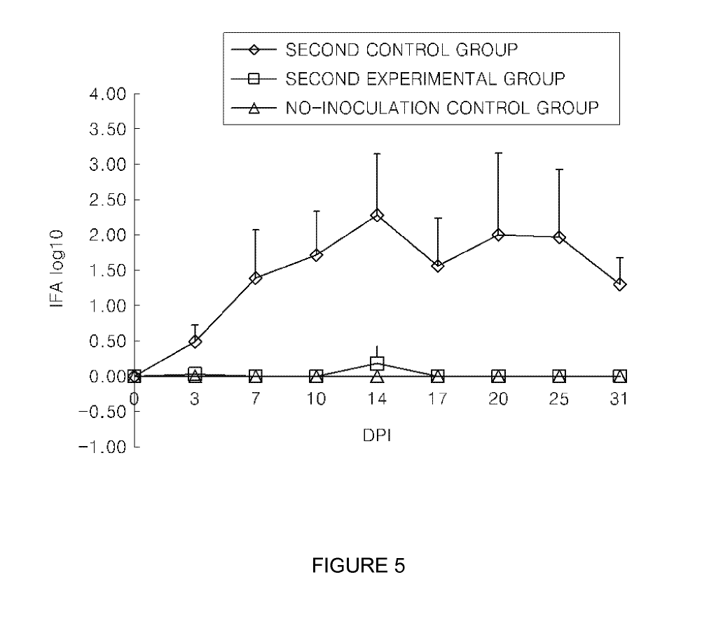

As a result of measuring the elution of the virus via the rectum, it was identified that the PCV2 elution via the excrement was significantly reduced in the first experimental group into which the vaccine was inoculated and the PCV2 was challenge-inoculated and in the second experimental group into which the vaccine was inoculated and PCV2 and PRRSV were challenge-inoculated when compared with the control groups. While the maximum amount of the eluted PCV2 was 10^{0.42} TCID₅₀/0.1 μl per day in the first and second experimental groups, the maximum amount of the eluted PCV2 was 10^{1.58} TCID₅₀/0.1 μl in the no-inoculation control group. In the first control group into which PCV2 was inoculated and the second control group into which PCV2 and PRRSV were inoculated, PCV2 was detected since the 3^{rd} day of the inoculation. While the maximum amount of the eluted PCV2 was 10^{1.95} TCID₅₀/0.1 μl per day in the first control group, the maximum amount of the eluted PCV2 was 10^{1.44} TCID₅₀/0.1 μl in the second control group. In the no-inoculation control group, no PCV2 elution was identified (FIGS. 4 and 5).

FIG. 4 is a graph illustrating PCV2 elution via excrement after PCV2 challenge-inoculation into young pigs. In FIG. 4, the x and y axes are defined with reference to FIG. 2.

FIG. 5 is a graph illustrating PCV2 elution via excrement after challenge-inoculation of PCV2 and PRRSV into young pigs. In FIG. 5, the x and y axes are defined with reference to FIG. 2.

As described above, in order to identify effectiveness of the vaccine including the attenuated porcine circovirus, pathogenicity and defense capability tests were conducted using pigs. As a result, it was observed that the vaccinated and challenge-inoculated groups did not show particular clinical symptoms compared with the non-vaccinated and challenge-inoculated groups. In addition, it was identified that the elution time of the virus via the nasal cavity or excrement of the vaccinated groups was shorter than that of the control group, and the amount of the eluted virus of the vaccinated groups was significantly less than that of the non-vaccinated groups. Accordingly, it was identified that the vaccine provides excellent defense capability in pigs.

### Example 6

### Outdoor Clinical Trials and Results

(6.1) Outdoor Clinical Trials

Vaccine including the attenuated porcine circovirus prepared according to an embodiment of the present invention was administered to pigs raised in outdoor farms to identify safety and effectiveness of the vaccine.

The vaccine was prepared as described above, and three outdoor farms were used for the trials. A first farm raised about 500 sows pigs, a second farm raised about 200 sows, and a third farm raised about 200 sows. The geographical locations of the three farms were different. Table 10 shows the number of pigs used for the trials.

TABLE 10

Young Pigs

| Group | Farm Name | Inoculation | Control |
|---|---|---|---|
| First group | Eden | 20/30 a | 20/20 |
| 2^{nd} group | Irae | 20/20 | 20/20 |
| 3^{rd} group | Garam | 20/20 | 20/20 |

| | |
|---|---|
| Total | 120/130 |

a: Number of pigs, blood of which is collected/number of pigs presented.

One μl of the vaccine was intramuscularly inoculated into each of 3 to 4-week old pigs (inoculation group). After about 2 weeks, the second inoculation of the vaccine was conducted in the same manner. Blood samples of the young pigs were collected before the vaccination (3-week old), after two weeks from the second inoculation (7-week old), after five weeks from the second inoculation (10-week old), after eight weeks from the second inoculation (13-week old), and after 11 weeks from the second inoculation (16-week old), and serum was isolated from their blood.

Clinical symptoms of the young pigs were observed as follows. First, topical and systematic symptoms were observed right after the inoculation of the vaccine.

During the trials, clinical symptoms of the vaccinated group and the control group were observed periodically according to the blood collection. Mainly, cough, appetite disorder, sneeze, mange, dyspnea, vomiting, high fever, bleeding (nose and mouth), lack of strength, general prostration, diarrhea, and the like were observed and the results were recorded. In order to identify outdoor effectiveness and safety of the vaccine, body temperature, weight, and antibody titer of the pigs were measured after the vaccination. The body temperatures of the pigs were measured three times a day (morning, lunch, and dinner) before the inoculation, after 4 hours from the inoculation, and the first and second days from the inoculation, and once a day from the 3^{rd} day to 7^{th} day. The weights of the pigs were measured at the ages of 3 weeks, 10 weeks, 13 weeks, and 16 weeks.

The PCV2 antibody titers were measured at the ages of 3 weeks, 7 weeks, 10 weeks, 13 weeks, and 16 weeks. As the antibody titer of the all pigs, blood of which was collected, PCV2 competitive ELISA antibody titer (Synbiotics) was measured. The PCV2 competitive ELISA antibody titer will be described below.

### PCV2 Ab Mono Blocking (PCV2 Competitive ELISA)

First, controls and samples were prepared. A vial was shaken before use, 100 μl of a negative control were added to A1 and A2 wells and 100 μl of a positive control were added to B1 and B2 wells. Then, the samples were diluted using a sample-diluting solution to a ratio of 1:100, and 100 μl of the samples was added to each well. Each sample was placed in one well, or placed in two wells and an average was obtained. The wells were covered with an adhesive film. The plate was cultivated at 37° C.±3° C. for 1 hour±5 minutes. The adhesive film was removed, and then the plate was washed four times with a washing solution diluted to a ratio of 1:10.

A conjugate was diluted to a ratio of 1:100 using a conjugate-diluting solution. 100 μl of the diluted conjugate was added to each well and the well was covered with a new adhesive film. The plate was cultivated at 37° C.±3° C. for 1 hour±5 minutes. The adhesive film was removed, and then the plate was washed four times with a washing solution diluted to a ratio of 1:10. Then, 100 μl of a substrate solution was added to each well, and the plate was slightly shaken. The plate was cultivated at room temperature for 30 minutes±5 minutes while blocking light. Then, 50 μl of a stop solution was added to each well, and the plate was slightly shaken. Optical density (OD) was measured at a wavelength of 450 nm.

The results were evaluated according to the following standards. The OD of the control was shown below.
OD range of the negative control >0.500
OD range of the positive control ≦0.300
Serum neutralizing antibody titer (SNc) was calculated according to Formula 2 below.

$$SNc = \frac{\begin{matrix}OD value for the sample - \\ OD value for the positive control\end{matrix}}{\begin{matrix}OD value for the negative control - \\ OD value for the positive control\end{matrix}} \qquad Formula 2$$

If the result is ≦0.50, the sample was judged to be positive. If the result is >0.50, the sample was judged to be negative.

In addition, death rates of the pigs used for the trials were measured, and PCV2 was detected by PCR, immunohistochemistry (IHC), and Salmonella test using PCR.

(6.2) Results of Outdoor Clinical Trials

(6.2.1) First Farm

The body temperatures of the vaccine-inoculated group and the control group were measured every day before the inoculation and for 7 days after the inoculation. As a result, all individual pigs of the inoculation group and the control group had normal body temperatures for 7 days.

Weights of the inoculation group and the control group were measured at the ages of 3 weeks (before the vaccine inoculation), 10 weeks, 13 weeks, and 16 weeks. Before the vaccine inoculation, the weight of the inoculation group was slightly less than that of the control group. However, at the age of 10 weeks, the weights of the inoculation group and the control group were similar to each other, and at the ages of 13 weeks and 16 weeks, the average weight of the inoculation group was slightly greater than that of the control group. Table 11 shows weights of the inoculation group and the control group (kg, n=20).

TABLE 11

| | Before | | | |
|---|---|---|---|---|
| | inoculation, | 10-week | 13-week | 16-week |
| | 3-week old | old | old | old |
| Vaccine | 8.57 | 20.40 | 31.29 | 52.19 |
| Inoculation group | | | | |
| Control group | 9.06 | 18.82 | 28.44 | 47.19 |

Blood samples of the pigs of the inoculation group and the control group were collected before the vaccine inoculation (3-week old), and at the ages of 7 weeks, 10 weeks, 13 weeks, and 16 weeks, and antibody titer of PCV2 was measured using ELISA and SN antibody titer was measured using IFA. In the inoculation group, an antibody positive rate significantly increased after two weeks from the second inoculation (7-week old) and the increased antibody positive rate was maintained until the age of 16 weeks. In the control group, the antibody positive rate increased or decreased during the trials and maintained to be lower than that of the inoculation group. The SN antibody titer of the control group was greater than that of the inoculation group before the inoculation (3-week old). However, at the age of 7 weeks, i.e., two weeks from the second inoculation, the SN antibody titer of the inoculation group was significantly greater than that of the control group (p<0.05). Table 12 shows PCV2 antibody titers of the inoculation group of the first farm and Table 13 shows PCV2 antibody titers of the control group of the first farm.

TABLE 12

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.39 | 8 | 0.16 | 64 | 0.22 | 32 | 0.13 | 64 | 0.33 | 16 |
| 2 | 0.33 | 16 | 0.19 | 64 | 0.20 | 32 | 0.21 | 43 | 0.21 | 32 |
| 3 | 0.41 | 4 | 0.14 | 64 | 0.32 | 16 | 0.13 | 64 | 0.21 | 32 |
| 4 | 0.29 | 16 | 0.11 | 64 | 0.21 | 32 | 0.16 | 64 | 0.13 | 64 |
| 5 | 0.39 | 4 | 0.13 | 64 | 0.21 | 32 | 0.22 | 32 | 0.23 | 32 |
| 6 | 0.35 | 8 | 0.20 | 64 | 0.13 | 64 | 0.29 | 16 | 0.26 | 32 |
| 7 | 0.39 | 2 | 0.32 | 16 | 0.16 | 64 | 0.22 | 32 | 0.22 | 32 |
| 8 | 0.31 | 16 | 0.08 | 128 | 0.22 | 32 | 0.20 | 32 | 0.29 | 16 |
| 9 | 0.25 | 16 | 0.16 | 64 | 0.20 | 32 | 0.32 | 16 | 0.24 | 16 |
| 10 | 0.29 | 16 | 0.22 | 32 | 0.28 | 16 | 0.21 | 32 | 0.26 | 16 |
| 11 | 0.30 | 16 | 0.20 | 32 | 0.22 | 32 | 0.20 | 32 | 0.36 | 8 |
| 12 | 0.33 | 16 | 0.32 | 16 | 0.26 | 32 | 0.19 | 32 | 0.23 | 32 |
| 13 | 0.31 | 16 | 0.21 | 32 | 0.19 | 32 | 0.25 | 32 | 0.31 | 32 |
| 14 | 0.40 | 4 | 0.11 | 64 | 0.13 | 64 | 0.22 | 32 | 0.21 | 16 |
| 15 | 0.41 | 2 | 0.13 | 64 | 0.21 | 32 | 0.20 | 32 | — | — |
| 16 | 0.39 | 4 | 0.16 | 64 | 0.13 | 64 | 0.32 | 16 | 0.35 | 16 |
| 17 | 0.41 | 2 | 0.22 | 32 | 0.16 | 64 | 0.26 | 16 | 0.22 | 32 |
| 18 | 0.29 | 16 | 0.20 | 64 | 0.22 | 32 | 0.28 | 32 | 0.36 | 16 |
| 19 | 0.39 | 4 | 0.32 | 16 | 0.20 | 32 | 0.22 | 32 | 0.30 | 32 |
| 20 | 0.35 | 8 | 0.08 | 64 | 0.18 | 32 | 0.20 | 32 | 0.31 | 16 |
| Average | 0.35 | 9.70 | 0.19 | 53.60 | 0.20 | 38.40 | 0.22 | 34.15 | 0.25 | 24.40 |

TABLE 13

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.28 | 32 | 0.39 | 8 | 0.36 | 16 | 0.36 | 8 | 0.35 | 8 |
| 2 | 0.25 | 32 | 0.41 | 4 | 0.37 | 16 | 0.37 | 16 | 0.39 | 2 |
| 3 | 0.29 | 16 | 0.29 | 16 | 0.27 | 32 | 0.14 | 64 | 0.30 | 16 |
| 4 | 0.30 | 16 | 0.39 | 8 | 0.16 | 64 | 0.32 | 16 | 0.23 | 32 |
| 5 | 0.33 | 16 | 0.35 | 8 | 0.31 | 32 | 0.32 | 16 | 0.33 | 16 |
| 6 | 0.31 | 16 | 0.36 | 8 | 0.25 | 32 | 0.31 | 32 | 0.24 | 32 |
| 7 | 0.36 | 16 | 0.35 | 8 | 0.29 | 16 | 0.33 | 16 | 0.35 | 8 |
| 8 | 0.37 | 8 | 0.39 | 4 | 0.26 | 32 | 0.31 | 16 | 0.36 | 8 |
| 9 | 0.27 | 32 | 0.31 | 16 | 0.33 | 16 | 0.35 | 8 | 0.25 | 32 |
| 10 | 0.21 | 32 | 0.25 | 32 | 0.34 | 16 | 0.28 | 16 | 0.30 | 16 |
| 11 | 0.32 | 16 | 0.29 | 16 | 0.28 | 32 | 0.22 | 32 | 0.36 | 8 |
| 12 | 0.41 | 4 | 0.30 | 16 | 0.21 | 32 | 0.26 | 16 | 0.37 | 8 |
| 13 | 0.38 | 8 | — | — | — | — | — | — | — | — |
| 14 | 0.26 | 32 | 0.23 | 32 | 0.20 | 32 | 0.36 | 8 | 0.44 | 0 |
| 15 | 0.37 | 8 | 0.31 | 16 | 0.16 | 64 | 0.40 | 4 | 0.29 | 16 |
| 16 | 0.27 | 32 | 0.25 | 32 | 0.37 | 16 | 0.24 | 32 | 0.41 | 2 |
| 17 | 0.21 | 32 | 0.29 | 32 | 0.34 | 16 | 0.30 | 16 | 0.33 | 16 |
| 18 | 0.32 | 16 | 0.26 | 32 | 0.25 | 32 | 0.38 | 8 | 0.31 | 32 |
| 19 | 0.30 | 16 | 0.33 | 16 | 0.26 | 16 | 0.33 | 16 | 0.20 | 32 |
| 20 | 0.38 | 8 | 0.31 | 16 | 0.20 | 32 | 0.34 | 16 | 0.31 | 16 |
| Average | 0.31 | 19.40 | 0.30 | 16.00 | 0.26 | 27.20 | 0.29 | 17.80 | 0.31 | 15.00 |

In the inoculation group, no pig died for 16 weeks after the vaccine was inoculated. In the control group, one pig died at the age of 7 weeks. Lymph nodes and lung were positive to PCV2 as a result of conducting PCR using a tissue sample of the dead pig of the control group as a template but negative to PCV2 in the tissue IHC staining.

During the trials, a Salmonella test was conducted. The dead pig was negative to Salmonella.

Clinical symptoms of the inoculation group and the control group were observed by the age of 16 weeks. While most of the pigs of the inoculation group showed normal conditions, some of the pigs of the control group showed PCV2 infection-associated symptoms such as depression, Watery diarrhea, rough hair, and atrophy. Table 14 shows clinical symptoms of the inoculation group, and Table 15 shows clinical symptoms of the control group.

TABLE 14

| | | No. of pigs | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 30 | 30 | 30 | 30 | 30 | 30 | 30 | 30 | 30 | 30 | 30 | 30 | 30 | 30 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 27 | 27 | 28 | 28 | 29 | 30 | 30 | 30 | 30 | 30 | 30 | 30 | 30 | 30 |
| | Intermittence | 3 | 3 | 2 | 2 | 1 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 30 | 30 | 30 | 30 | 30 | 30 | 30 | 30 | 30 | 30 | 30 | 30 | 30 | 30 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 30 | 30 | 30 | 30 | 30 | 30 | 30 | 30 | 30 | 30 | 30 | 30 | 30 | 30 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 30 | 30 | 30 | 30 | 29 | 29 | 30 | 30 | 30 | 30 | 29 | 29 | 30 | 30 |
| | Watery diarrhea | 0 | 0 | 0 | 0 | 1 | 1 | 0 | 0 | 0 | 0 | 1 | 1 | 0 | 0 |
| | Diarrhea | 0 | 0 | 0 | 0 | 0 | 0 | 0 | 0 | 0 | 0 | 0 | 0 | 0 | 0 |

TABLE 15

| | | No. of pigs | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 20 | 20 | 20 | 18 | 19 | 19 | 19 | 19 | 19 | 19 | 19 | 19 | 19 | 19 |
| | Increase | 0 | 0 | 0 | 2 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 20 | 20 | 20 | 18 | 18 | 19 | 19 | 18 | 17 | 17 | 19 | 19 | 19 | 19 |
| | Intermittence | 0 | 0 | 0 | 2 | 1 | 0 | 0 | 1 | 2 | 2 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 20 | 20 | 20 | 20 | 19 | 19 | 19 | 19 | 19 | 19 | 19 | 19 | 19 | 19 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 20 | 20 | 20 | 20 | 19 | 19 | 19 | 19 | 19 | 19 | 19 | 19 | 19 | 19 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 20 | 20 | 20 | 20 | 19 | 19 | 17 | 17 | 18 | 17 | 19 | 19 | 17 | 17 |
| | Watery diarrhea | 0 | 0 | 0 | 0 | 0 | 0 | 2 | 2 | 1 | 2 | 0 | 0 | 2 | 2 |
| | Diarrhea | 0 | 0 | 0 | 0 | 0 | 0 | 0 | 0 | 0 | 0 | 0 | 0 | 0 | 0 |

(6.2.2) Second Farm

The body temperatures of the vaccine-inoculated group and the control group were measured every day before the inoculation and for 7 days after the inoculation. All individual pigs of the inoculation group and the control group showed normal body temperatures for 7 days.

Weights of the inoculation group and the control group were measured at the ages of 3 weeks (before the vaccine inoculation), 10 weeks, 13 weeks, and 16 weeks. Until the age of 10 weeks, the weights of the inoculation group and the control group were similar to each other, and at the ages of 13 weeks and 16 weeks, the average weight of the inoculation group was slightly greater than that of the control group. Table 16 shows weights of the inoculation group and the control group (kg, n=20).

TABLE 16

| | Before | | | |
|---|---|---|---|---|
| | inoculation, | 10-week | 13-week | 16-week |
| | 3-week old | old | old | old |
| Vaccine | 8.57 | 20.04 | 31.93 | 51.82 |
| inoculation group | | | | |
| Control group | 8.79 | 19.15 | 29.08 | 49.32 |

Blood samples of the pigs of the inoculation group and the control group were collected before the vaccine inoculation (3-week old), and at the ages of 10 weeks, 13 weeks, and 16 weeks, and antibody titer of PCV2 was measured using ELISA and SN antibody titer was measured using IFA. In the inoculation group, an antibody titer was significantly increased after two weeks from the second inoculation (7-week old) and the increased antibody titer maintained until the age of 16 weeks. In the control group, the antibody positive rate and the antibody titer gradually increased to be similar to those of the inoculation group at the age of 13 weeks. The SN antibody titers of the inoculation group and the control group were seronegative (≦1:10). However, after two weeks from the second inoculation (7-week old), an average SN antibody titer of the inoculation group was 1:25, and an average SN antibody titer of the control group was 1:10 to exhibit a significant difference (p<0.05), and the difference increased until the age of 10 weeks. Since the antibody titer of the control group increased from the age of 13 weeks so as to be similar to that of the inoculation group. Table 17 shows PCV2 antibody titer of the inoculation group of the second farm and Table 18 shows PCV2 antibody titer of the control group of the second farm.

TABLE 17

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.48 | 4 | 0.34 | 4 | 0.39 | 4 | 0.23 | 16 | 0.15 | 64 |
| 2 | 0.49 | 4 | 0.25 | 16 | 0.25 | 16 | 0.29 | 16 | 0.28 | 16 |
| 3 | 0.38 | 4 | 0.22 | 32 | 0.11 | 64 | 0.20 | 32 | 0.31 | 16 |
| 4 | 0.55 | 0 | 0.31 | 16 | 0.15 | 64 | 0.13 | 64 | 0.28 | 16 |
| 5 | 0.45 | 2 | 0.39 | 4 | 0.15 | 64 | 0.17 | 64 | 0.10 | 128 |
| 6 | 0.56 | 2 | 0.27 | 32 | 0.26 | 32 | 0.20 | 32 | 0.14 | 64 |
| 7 | 0.56 | 0 | 0.15 | 128 | 0.07 | 128 | 0.08 | 128 | 0.09 | 128 |
| 8 | 0.39 | 4 | 0.25 | 32 | 0.18 | 64 | 0.29 | 16 | 0.28 | 16 |
| 9 | 0.45 | 4 | 0.38 | 8 | 0.11 | 64 | 0.13 | 32 | 0.12 | 64 |
| 10 | 0.35 | 8 | 0.31 | 16 | 0.08 | 64 | 0.10 | 64 | 0.09 | 64 |
| 11 | 0.30 | 16 | 0.19 | 64 | 0.20 | 32 | 0.26 | 16 | 0.13 | 64 |
| 12 | 0.49 | 4 | 0.28 | 32 | 0.34 | 4 | 0.24 | 16 | 0.26 | 32 |
| 13 | 0.23 | 32 | 0.30 | 16 | 0.11 | 64 | 0.10 | 64 | 0.15 | 64 |
| 14 | 0.26 | 32 | 0.16 | 64 | 0.23 | 16 | 0.14 | 64 | 0.20 | 32 |
| 15 | 0.34 | 8 | 0.40 | 2 | 0.14 | 32 | 0.28 | 16 | 0.30 | 16 |
| 16 | 0.38 | 4 | 0.39 | 4 | 0.18 | 64 | 0.17 | 64 | 0.14 | 64 |
| 17 | 0.46 | 2 | 0.34 | 4 | 0.15 | 64 | 0.19 | 32 | 0.20 | 32 |
| 18 | 0.50 | 2 | 0.25 | 16 | 0.29 | 16 | 0.22 | 16 | 0.20 | 64 |
| 19 | 0.41 | 2 | 0.48 | 2 | 0.37 | 8 | 0.21 | 32 | 0.18 | 64 |
| 20 | 0.50 | 2 | 0.26 | 16 | 0.17 | 64 | 0.25 | 16 | 0.19 | 64 |
| Average | 0.42 | 6.80 | 0.30 | 25.40 | 0.20 | 46.40 | 0.19 | 40.00 | 0.19 | 53.60 |

TABLE 18

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.42 | 2 | 0.35 | 4 | 0.45 | 2 | 0.28 | 16 | 0.29 | 16 |
| 2 | 0.53 | 0 | 0.50 | 0 | 0.51 | 0 | 0.15 | 64 | 0.18 | 64 |
| 3 | 0.45 | 0 | 0.49 | 0 | 0.44 | 0 | 0.29 | 16 | 0.25 | 32 |
| 4 | 0.55 | 0 | 0.41 | 2 | 0.41 | 2 | 0.42 | 2 | 0.30 | 16 |
| 5 | 0.48 | 0 | 0.39 | 4 | 0.38 | 4 | 0.18 | 64 | 0.19 | 64 |
| 6 | 0.25 | 16 | 0.45 | 2 | 0.37 | 4 | 0.31 | 8 | 0.18 | 64 |
| 7 | 0.34 | 8 | 0.18 | 64 | 0.19 | 32 | 0.12 | 64 | 0.16 | 64 |
| 8 | 0.21 | 32 | 0.29 | 8 | 0.28 | 16 | 0.08 | 64 | 0.14 | 64 |
| 9 | 0.28 | 16 | 0.24 | 16 | 0.25 | 32 | 0.19 | 32 | 0.13 | 64 |
| 10 | 0.30 | 8 | 0.37 | 4 | 0.39 | 4 | 0.20 | 32 | 0.21 | 32 |
| 11 | 0.52 | 0 | 0.49 | 0 | 0.39 | 2 | 0.20 | 32 | 0.18 | 64 |
| 12 | 0.45 | 2 | 0.42 | 2 | 0.14 | 64 | 0.13 | 64 | 0.15 | 64 |
| 13 | 0.40 | 2 | 0.32 | 4 | 0.38 | 4 | 0.15 | 64 | 0.19 | 32 |
| 14 | 0.39 | 4 | 0.34 | 4 | 0.37 | 4 | 0.18 | 64 | 0.20 | 32 |
| 15 | 0.47 | 0 | 0.34 | 8 | 0.39 | 4 | 0.16 | 64 | 0.17 | 64 |
| 16 | 0.29 | 8 | 0.37 | 4 | 0.26 | 16 | 0.20 | 32 | 0.18 | 64 |
| 17 | 0.13 | 64 | 0.17 | 64 | 0.12 | 64 | 0.26 | 32 | 0.21 | 32 |
| 18 | 0.43 | 2 | 0.43 | 2 | 0.45 | 2 | 0.22 | 32 | 0.27 | 16 |
| 19 | 0.48 | 2 | 0.28 | 16 | 0.29 | 16 | 0.20 | 32 | 0.28 | 16 |
| 20 | 0.49 | 0 | 0.49 | 0 | 0.47 | 0 | 0.12 | 64 | 0.06 | 128 |
| Average | 0.39 | 8.30 | 0.36 | 10.40 | 0.34 | 13.60 | 0.21 | 42.10 | 0.20 | 49.60 |

No pig died from the inoculation group and the control group.

Clinical symptoms of the inoculation group and the control group were observed by the age of 16 weeks. While most of the pigs of the inoculation group showed normal conditions, about three pigs of the control group showed watery diarrhea and rough hair. Table 19 shows clinical symptoms of the inoculation group, and Table 20 shows clinical symptoms of the control group.

TABLE 19

| | | No. of individuals having clinical symptoms | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 20 | 20 | 20 | 20 | 20 | 20 | 20 | 20 | 20 | 20 | 20 | 20 | 20 | 20 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 20 | 20 | 20 | 20 | 20 | 20 | 20 | 20 | 20 | 20 | 20 | 20 | 20 | 20 |
| | Intermittence | 0 | 0 | 0 | 0 | 0 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 20 | 20 | 20 | 20 | 20 | 20 | 20 | 20 | 20 | 20 | 20 | 20 | 20 | 20 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 20 | 20 | 20 | 20 | 20 | 20 | 20 | 20 | 20 | 20 | 20 | 20 | 20 | 20 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 20 | 20 | 20 | 20 | 20 | 20 | 20 | 20 | 20 | 20 | 20 | 20 | 20 | 20 |
| | Watery diarrhea | 0 | 0 | 0 | 0 | 0 | 0 | 0 | 0 | 0 | 0 | 0 | 0 | 0 | 0 |
| | Diarrhea | 0 | 0 | 0 | 0 | 0 | 0 | 0 | 0 | 0 | 0 | 0 | 0 | 0 | 0 |

TABLE 20

| | | No. of individuals having clinical symptoms | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 20 | 20 | 20 | 20 | 20 | 20 | 20 | 20 | 20 | 20 | 20 | 20 | 20 | 20 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 20 | 20 | 20 | 20 | 20 | 20 | 20 | 20 | 20 | 20 | 20 | 20 | 20 | 20 |
| | Intermittence | 0 | 0 | 0 | 0 | 0 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 20 | 20 | 20 | 20 | 20 | 20 | 20 | 20 | 20 | 20 | 20 | 20 | 20 | 20 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 20 | 20 | 20 | 20 | 20 | 20 | 20 | 20 | 20 | 20 | 20 | 20 | 20 | 20 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 20 | 20 | 20 | 20 | 19 | 19 | 19 | 20 | 20 | 20 | 20 | 20 | 20 | 20 |
| | Watery diarrhea | 0 | 0 | 0 | 0 | 1 | 1 | 1 | 0 | 0 | 0 | 0 | 0 | 0 | 0 |
| | Diarrhea | 0 | 0 | 0 | 0 | 0 | 0 | 0 | 0 | 0 | 0 | 0 | 0 | 0 | 0 |

(6.2.3) Third Farm

Body temperatures of the vaccine-inoculated group and the control group were measured every day before the inoculation and for 7 days after the inoculation. All individual pigs of the inoculation group and the control group had normal body temperatures for 7 days.

Weights of the inoculation group and the control group were measured at the ages of 3 weeks (before the vaccine inoculation), 10 weeks, 13 weeks, and 16 weeks. After the age of 10 weeks, an average weight of the pigs in the inoculation group was greater than that of the control group. Table 21 shows weights of the inoculation group and the control group (kg, n=20).

TABLE 21

| | Before | | | |
|---|---|---|---|---|
| | inoculation, | 10-week | 13-week | 16-week |
| | 3-week old | old | old | old |
| Vaccine | 4.89 | 27.66 | 40.16 | 53.26 |
| inoculation group | | | | |
| Control group | 4.90 | 25.62 | 36.81 | 47.91 |

Blood samples of the pigs of the inoculation group and the control group were collected before the vaccine inoculation (3-week old), and at the ages of 10 weeks, 13 weeks, and 16 weeks, and antibody titer of PCV2 was measured using ELISA and SN antibody titer was measured using IFA. In the inoculation group, an antibody positive rate and an antibody titer was significantly increased after two weeks from the second inoculation (7-week old) and the increased antibody positive rate and antibody titer maintained until the age of 16 weeks. In the control group, the antibody positive rate gradually increased. However, a negative individual was identified at the age of 16 weeks. The SN antibody titers of the inoculation group and the control group were seronegative (≦1:10). However, after the second inoculation, an average SN antibody titer of the inoculation group was 1:33), and an average SN antibody titer of the control group was 1:15 to exhibit a significant difference (p<0.05). Then, this significant difference was maintained until the age of 16 weeks. Table 22 shows PCV2 antibody titer of the inoculation group of the third farm and Table 23 shows PCV2 antibody titer of the control group of the third farm.

TABLE 22

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.25 | 32 | 0.35 | 16 | 0.28 | 32 | 0.25 | 32 | 0.20 | 64 |
| 2 | 0.40 | 4 | 0.42 | 2 | 0.26 | 32 | 0.28 | 32 | 0.21 | 32 |
| 3 | 0.49 | 0 | 0.28 | 32 | 0.27 | 32 | 0.21 | 64 | 0.24 | 32 |
| 4 | 0.53 | 0 | 0.19 | 64 | 0.14 | 64 | 0.14 | 64 | 0.16 | 32 |
| 5 | 0.47 | 0 | 0.27 | 32 | 0.19 | 64 | 0.15 | 64 | 0.26 | 16 |
| 6 | 0.41 | 2 | 0.40 | 2 | 0.35 | 16 | 0.29 | 16 | 0.23 | 32 |
| 7 | 0.51 | 0 | 0.15 | 64 | 0.14 | 64 | 0.13 | 64 | 0.21 | 32 |
| 8 | 0.47 | 0 | 0.23 | 32 | 0.26 | 32 | 0.28 | 32 | 0.20 | 32 |
| 9 | 0.49 | 0 | 0.24 | 32 | 0.21 | 32 | 0.23 | 32 | 0.27 | 32 |
| 10 | 0.52 | 0 | 0.18 | 64 | 0.17 | 64 | 0.19 | 64 | 0.16 | 64 |
| 1 | 0.45 | 2 | 0.38 | 8 | 0.28 | 32 | 0.21 | 64 | 0.26 | 32 |
| 12 | 0.42 | 2 | 0.47 | 0 | 0.46 | 2 | 0.31 | 16 | 0.20 | 32 |
| 13 | 0.28 | 16 | 0.29 | 16 | 0.24 | 32 | 0.18 | 64 | 0.21 | 64 |
| 14 | 0.46 | 0 | 0.40 | 2 | 0.42 | 2 | 0.36 | 16 | 0.26 | 32 |
| 15 | 0.50 | 0 | 0.27 | 32 | 0.29 | 16 | 0.24 | 32 | 0.25 | 32 |
| 16 | 0.54 | 0 | 0.14 | 64 | 0.13 | 64 | 0.17 | 64 | 0.19 | 64 |
| 17 | 0.38 | 4 | 0.46 | 2 | 0.19 | 32 | 0.11 | 64 | 0.14 | 64 |
| 18 | 0.45 | 2 | 0.24 | 32 | 0.16 | 64 | 0.13 | 64 | 0.18 | 64 |
| 19 | 0.49 | 0 | 0.25 | 32 | 0.21 | 32 | 0.21 | 32 | 0.21 | 32 |
| 20 | 0.52 | 0 | 0.09 | 128 | 0.10 | 64 | 0.12 | 64 | 0.18 | 64 |
| Average | 0.45 | 3.20 | 0.30 | 32.80 | 0.24 | 38.60 | 0.21 | 47.20 | 0.21 | 42.40 |

TABLE 23

| | Before | | | | | | | | | |
|---|---|---|---|---|---|---|---|---|---|---|
| | inoculation, | | | | | | | | | |
| | 3-week old | | 7-week old | | 10-week old | | 13-week old | | 16-week old | |
| No. | ELISA | SN | ELISA | SN | ELISA | SN | ELISA | SN | ELISA | SN |
| 1 | 0.51 | 0 | 0.47 | 0 | 0.38 | 8 | 0.39 | 8 | 0.30 | 32 |
| 2 | 0.50 | 0 | 0.54 | 0 | 0.47 | 0 | 0.39 | 8 | 0.31 | 16 |
| 3 | 0.40 | 4 | 0.37 | 8 | 0.34 | 16 | 0.36 | 8 | 0.24 | 32 |
| 4 | 0.54 | 0 | 0.45 | 2 | 0.32 | 16 | 0.31 | 16 | 0.34 | 16 |
| 5 | 0.27 | 32 | 0.32 | 16 | 0.24 | 32 | 0.22 | 32 | 0.21 | 32 |
| 6 | 0.41 | 2 | 0.40 | 4 | 0.21 | 32 | 0.20 | 32 | 0.14 | 64 |
| 7 | 0.41 | 4 | 0.35 | 16 | 0.37 | 16 | 0.39 | 8 | 0.40 | 8 |
| 8 | 0.38 | 8 | 0.34 | 16 | 0.24 | 32 | 0.26 | 32 | 0.19 | 64 |
| 9 | 0.42 | 2 | 0.41 | 4 | 0.45 | 2 | 0.46 | 2 | 0.44 | 2 |
| 10 | 0.26 | 32 | 0.25 | 32 | 0.22 | 32 | 0.19 | 64 | 0.20 | 32 |
| 1 | 0.48 | 0 | 0.42 | 4 | 0.44 | 2 | 0.41 | 4 | 0.43 | 2 |
| 12 | 0.53 | 0 | 0.51 | 0 | 0.50 | 0 | 0.51 | 0 | 0.50 | 0 |
| 13 | 0.47 | 0 | 0.48 | 0 | 0.51 | 0 | 0.47 | 0 | 0.49 | 0 |
| 14 | 0.34 | 16 | 0.35 | 16 | 0.32 | 16 | 0.30 | 32 | 0.31 | 16 |
| 15 | 0.47 | 2 | 0.41 | 4 | 0.43 | 2 | 0.24 | 32 | 0.21 | 64 |
| 16 | 0.33 | 16 | 0.34 | 32 | 0.12 | 64 | 0.14 | 64 | 0.12 | 64 |
| 17 | 0.44 | 2 | 0.21 | 64 | 0.24 | 32 | 0.20 | 32 | 0.21 | 64 |
| 18 | 0.51 | 0 | 0.14 | 64 | 0.15 | 64 | 0.21 | 32 | 0.18 | 64 |
| 19 | 0.43 | 2 | 0.37 | 16 | 0.39 | 8 | 0.33 | 32 | 0.34 | 16 |
| 20 | 0.52 | 0 | 0.55 | 0 | 0.47 | 0 | 0.51 | 0 | 0.50 | 0 |
| Average | 0.43 | 6.10 | 0.37 | 14.90 | 0.30 | 18.70 | 0.24 | 21.90 | 0.22 | 29.40 |

During the trials, there was no dead pig in the inoculation group and the control group.

Clinical symptoms of the inoculation group and the control group were observed by the age of 16 weeks. While most of the pigs of the inoculation group and the control group showed normal conditions, some pigs of the control group showed cough and watery diarrhea, but recovered after treatment with antibiotics. Table 24 shows clinical symptoms of the inoculation group, and Table 25 shows clinical symptoms of the control group.

TABLE 24

| | | No. of individuals having clinical symptoms | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 20 | 20 | 20 | 20 | 20 | 20 | 20 | 20 | 20 | 20 | 20 | 20 | 20 | 20 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 20 | 20 | 20 | 20 | 20 | 20 | 20 | 20 | 20 | 20 | 20 | 20 | 20 | 20 |
| | Intermittence | 0 | 0 | 0 | 0 | 0 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 20 | 20 | 20 | 20 | 20 | 20 | 20 | 20 | 20 | 20 | 20 | 20 | 20 | 20 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 20 | 20 | 20 | 20 | 20 | 20 | 20 | 20 | 20 | 20 | 20 | 20 | 20 | 20 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 20 | 20 | 20 | 20 | 20 | 20 | 20 | 20 | 20 | 20 | 20 | 20 | 20 | 20 |
| | Watery diarrhea | 0 | 0 | 0 | 0 | 0 | 0 | 0 | 0 | 0 | 0 | 0 | 0 | 0 | 0 |
| | Diarrhea | 0 | 0 | 0 | 0 | 0 | 0 | 0 | 0 | 0 | 0 | 0 | 0 | 0 | 0 |

TABLE 25

| | | No. of individuals having clinical symptoms | | | | | | | | | | | | | |
|---|---|---|---|---|---|---|---|---|---|---|---|---|---|---|---|
| | | week | | | | | | | | | | | | | |
| | | 3 | 4 | 5 | 6 | 7 | 8 | 9 | 10 | 11 | 12 | 13 | 14 | 15 | 16 |
| Breathing | Normal | 20 | 20 | 20 | 20 | 20 | 20 | 20 | 20 | 20 | 20 | 20 | 20 | 20 | 20 |
| | Increase | 0 | 0 | 0 | 0 | 0 | 0 | 0 | 0 | 0 | 0 | 0 | 0 | 0 | 0 |
| | Opening mouth | 0 | 0 | 0 | 0 | 0 | 0 | 0 | 0 | 0 | 0 | 0 | 0 | 0 | 0 |
| Cough | Normal | 20 | 20 | 19 | 18 | 19 | 20 | 20 | 20 | 20 | 20 | 20 | 20 | 20 | 20 |
| | Intermittence | 0 | 0 | 1 | 2 | 1 | 0 | 0 | 0 | 0 | 0 | 0 | 0 | 0 | 0 |
| | Severe | 0 | 0 | 0 | 0 | 0 | 0 | 0 | 0 | 0 | 0 | 0 | 0 | 0 | 0 |
| Skin rashes | Normal | 20 | 20 | 20 | 20 | 20 | 20 | 20 | 20 | 20 | 20 | 20 | 20 | 20 | 20 |
| | Spots | 0 | 0 | 0 | 0 | 0 | 0 | 0 | 0 | 0 | 0 | 0 | 0 | 0 | 0 |
| | Jaundice | 0 | 0 | 0 | 0 | 0 | 0 | 0 | 0 | 0 | 0 | 0 | 0 | 0 | 0 |
| Motility | Normal | 20 | 20 | 20 | 20 | 20 | 20 | 20 | 20 | 20 | 20 | 20 | 20 | 20 | 20 |
| | Abnormal | 0 | 0 | 0 | 0 | 0 | 0 | 0 | 0 | 0 | 0 | 0 | 0 | 0 | 0 |
| | Daudication | 0 | 0 | 0 | 0 | 0 | 0 | 0 | 0 | 0 | 0 | 0 | 0 | 0 | 0 |
| Diarrhea | Normal | 20 | 20 | 20 | 20 | 20 | 18 | 19 | 19 | 20 | 20 | 20 | 20 | 20 | 20 |
| | Watery diarrhea | 0 | 0 | 0 | 0 | 0 | 2 | 1 | 1 | 0 | 0 | 0 | 0 | 0 | 0 |
| | Diarrhea | 0 | 0 | 0 | 0 | 0 | 0 | 0 | 0 | 0 | 0 | 0 | 0 | 0 | 0 |

(6.3) Conclusion

The outdoor clinical trials were conducted to determine the safety and immunogenicity of the vaccine including the attenuated porcine circovirus according to an embodiment of the present invention for outdoor farms.

130 young pigs from three different outdoor farms were used for the trials, and 1 μl of the immunogenic composition was intramuscularly inoculated twice at the age of 3 weeks and at the age of 5 weeks. As a result of observing clinical symptoms after the inoculation, there was no topical and systematical symptom that is commonly observed after 1 to 2 hours after the inoculation. In addition, clinical symptoms such as dyspnea, vomiting, bleeding, high fever, and general prostration were not observed during the trials. Even though some of the pigs exhibited appetite disorder but recovered within several days.

When the overall antibody titer after the second inoculation was measured from the three outdoor farms, a sero-conversion rate of the antibody against the circovirus was equal to or greater than 90% after the second inoculation compared to the antibody titer before the inoculation, thereby exhibiting excellent immunogenicity.

Natural cumulative death rate was about zero until the age of 16 weeks from the vaccine inoculation in the inoculation group and the control group. The weight gain rate of the vaccinated group was greater than that of the control group, thereby exhibiting a big difference of average weights between the pigs having the same age. According to the results described above, safety and defense capability of the vaccine in outdoor farms were sufficiently proved.

### Example 7

### Comparative Experiment

Effectiveness of the vaccine including the attenuated porcine circovirus according to the present invention was evaluated by comparison with a commercially available product. Ingelvac CircoFlex™ 309-129, 30991-kr2 (Boehringer Ingelhein GmbH, Korea) was used for the comparison. Ingelvac CircoFlex™ includes purified circovirus antigen (PCV™) and an excipient of Impran FLEX™ that is an aqueous polymer.

Three-week old young pigs from the first farm were used for the comparative experiment. 1 ml of the immunogenic composition according to the present invention was intramuscularly inoculated into behind the base of the ear of 3-week old pigs and 5-week old pigs to prepare group A, and 1 ml of Ingelvac CircoFlex™ was intramuscularly inoculated into behind the base of the ear of 3-week old pigs to prepare group B. Non-vaccinated control group was prepared as group C (Table 26).

TABLE 26

| | No. of | |
|---|---|---|
| Group | pigs | Vaccine inoculation |
| A | 750 | Vaccine of the present invention intramuscular |
| | | inoculation, twice at the ages of 3-week and |
| | | 5-week old |
| B | 98 | Ingelvac CircoFlex, intramuscular inoculation once |
| | | at the age of 3-week old |
| C | 49 | Non-Vaccinated |

Since Ingelvac CircoFlex™ does not have serological changes, weaning number, weights of 7-week old pigs, number of marketing pigs, and death rate after the vaccine inoculation were recorded and effects were compared.

While the weaning numbers of groups A, B, and C were similar, the shipping numbers of the experimental groups A and B were greater than that of group C. In particular, the shipping number of group A according to the present invention was greater than that of group B (Ingelvac CircoFlex™) (Table 27). Furthermore, an average weight of the 7-week old pigs of groups A and B was greater than that of group C, and average weights of groups A and B were similar to each other (Table 27). Table 27 shows weaning number (rate), average weight of 7-week old pigs (n=20), and shipping number (rate).

TABLE 27

| | Weaning number | Shipping number | Average weight of pigs |
|---|---|---|---|
| Group | (weaning rate¹) | (shipping rate²) | at the age of 7 weeks |
| A | 734(97.8) | 645(86.0) | 15.9 ± 0.9 |
| B | 95(96.9) | 74(75.5) | 15.0 ± 1.1 |
| C | 47(95.9) | 25(51.0) | 14.3 ± 1.7 |

¹Weaning rate = weaning number/number of animals × 100
²Shipping rate = shipping number/number of animals × 100

As described above, when the vaccine according to the present invention and a commercially available Ingelvac CircoFlex™ having similar effect were applied to farms for a predetermined period of time, the vaccine according to the present invention showed greater average weights of 7-week old pigs and shipping rates than Ingelvac CircoFlex™.

As demonstrated herein, PCV2 GC0504 and PCV2 GC0505 according to according to an embodiment of the present invention has high replication ability in host cells. The immunogenic composition according to an embodiment of the present invention has immunogenicity against at least one selected from the group consisting of various PCV2a and PCV2b. According to the method of producing attenuated circovirus according to an embodiment of the present invention, attenuated circovirus may be produced. According to a method of treating or preventing circovirus infection-associated diseases in animals, infection by at least one selected from the group consisting of PCV2a and PCV2b may be efficiently prevented or treated.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, assay methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. The invention illustratively described herein suitably may be practiced in the absence of any element or elements, limitation or limitations which is not specifically disclosed herein. Headings are used for convenience only.

All publications referred to herein are incorporated herein to the extent not inconsistent herewith. Some references provided herein are incorporated by reference to provide details of additional uses of the invention. All patents and publications mentioned in the specification are indicative of the levels of skill of those skilled in the art to which the invention pertains. References cited herein are incorporated by reference herein in their entirety to indicate the state of the art as of their filing date and it is intended that this information can be employed herein, if needed, to exclude specific embodiments that are in the prior art. For example, when a compound is claimed, it should be understood that compounds known in the art including the compounds disclosed in the references disclosed herein are not intended to be included in the claim.

What is claimed is:

1. A composition for treating or preventing circovirus infection in animals, the composition comprising at least on attenuated virus selected from the group consisting of attenuated circovirus PCV2 GC0504 and PCV2 GC0505.

2. The composition of claim 1, wherein PCV2 GC0504 comprises a genome having a nucleotide sequence of SEQ ID NO: 1.

3. The composition of claim 1, wherein PCV2 GC0505 comprises a genome having a nucleotide sequence of SEQ ID NO: 4.

4. The composition of claim 1, wherein PCV2 GC0504 and PCV2 GC0505 are attenuated by the reaction with formaldehyde.

5. The composition of claim 1, wherein the amounts of PCV2 GC0504 and PCV2 GC0505 are respectively in the range of 10^{4.5} TCID₅₀ to 10^{6.5} TCID₅₀ at a single dose.

6. The composition of claim 1, wherein the circovirus used to prevent and treat circovirus infection is selected from the group consisting of PCV2a and PCV2b.

7. The composition of claim 1, wherein the circovirus used to prevent and treat circovirus infection comprises PCV2a and PCV2b.

8. The composition of claim 1, further comprising a veterinarily acceptable excipient.

9. The composition of claim 8, wherein the excipient is montanide gel.

10. The composition of claim 8, wherein the amount of the excipient is in the range of 5 to 15% based on the total weight of the composition.

11. The composition of claim 1, wherein the animal is selected from the group consisting of pig, mouse, and guinea pig.

12. A method of producing attenuated circovirus, the method comprising reacting at least one virus selected from the group consisting of circovirus PCV2 GC0504 and PCV2 GC0505 with formalin to prepare attenuated circovirus.

13. The method of claim 12, wherein the amount of the formalin is in the range of 0.2 to 0.5 wt % based on the total weight of the reaction solution.

14. A method of treating or preventing circovirus infection in animals, the method comprising administering the composition according to any one of claims 1 in an effective amount into the animals.

15. The method of claim 14, wherein the animal is selected from the group consisting of pig, mouse, and guinea pig.

16. The method of claim 14, wherein the composition is orally or parenterally administered.

17. The method of claim 14, wherein the composition is injected.

18. Circovirus PCV2 GC0504 comprising a genome having a nucleotide sequence of SEQ ID NO: 1.

19. Circovirus PCV2 GC0505 comprising a genome having a nucleotide sequence of SEQ ID NO: 4.


SEQUENCE LISTING (8 sequences)

SEQ ID NO: 1 - DNA, 1768 bp, Porcine Circovirus type 2
  CDS (51)..(992): GC0504 positive strand, replicase protein
accagcgcacttcggcagcggcagcacctcggcagcacctcagcagcaacatgcccagcaagaagaatggaagaagcgggccccaaccacataaaaggtgggtgttcacgctgaataatccttccgaagacgagcgcaagaaaatacgggagctcccaatctccctgtttgattattttattgttggcgaggagggtaatgaggaaggacgaacacctcacctccaggggttcgctaattttgtgaagaagcaaacttttaataaagtgaagtggtatttgggtgcccgctgccacatcgagaaagccaaaggaactgatcagcagaataaagaatattgcagtaaagaaggcaacttacttattgaatgtggagctcctcgatctcaaggacaacggagtgacctgtctactgctgtgagtaccttgttggagagcgggagtctggtgaccgttgcagagcagcaccctgtaacgtttgtcagaaatttccgcgggccggctgaacttttgaaagtgagcgggaaaatgcagaagcgtgattggaagaccaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccgtgggatgacctactgagactgtgtgatcgatatccattgactgtagagactaaaggtggaactgtaccttttttggcccgcagtattctgattaccagcaatcagaccccgttggaatggtactcctcaactgctgtcccagctgtagaagctctctatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcatttagggtttaagtggggggtctttaagattaaattctctgaattgtacatacatggttacacggatattgtagtcctggtcgtatatactgttttcgaacgcagtgccgaggcctacgtggtccacatttctagaggtttgtagcctcagccaaagctgattccttttgttatttggttggaagtaatcaatagtggagtcaagaacaggtttgggtgtgaagtaacgggagtggtaggagaagggttgggggattgtatggcgggaggagtagtttacatatgggtcataggtttgggcattgacctttggtacaaagttatcatctagaataacagcagtggagcccactcccctatcaccctgggtgatgggggagcagggccaggattcaaccttaacctttcttattctgtagtattcagagggtatagagattttgttggtcccccctcccgggggaacaaagtcgtcaagattaaatctcatcatgtccaccgcccaggagggcgttctgactgtggtagccttgacagtatatccgaaggtgcgggagaggcgggtgttgaagatgccatttttccttctccaacggtagcggtggcgggggtggacgagccaggggcggcggcggaggatctggccaagatggctgcgggggcggtgtcttcttcttcggtaacgcctccttggatacgtcatagctgaaaacgaaagaagtgcgctgtaagtatt

SEQ ID NO: 2 - protein, 314 aa, Porcine Circovirus type 2
  GC0504
MPSKKNGRSGPQPHKRWVFTLNNPSEDERKKIRELPISLFDYFIVGEEGNEEGRTPHLQGFANFVKKQTFNKVKWYLGARCHIEKAKGTDQQNKEYCSKEGNLLIECGAPRSQGQRSDLSTAVSTLLESGSLVTVAEQHPVTFVRNFRGPAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKNATEQSTEEGGQFVTLSPPCPEFPYEINY

SEQ ID NO: 3 - protein, 233 aa, Porcine Circovirus type 2
  GC0504
MTYPRRRYRRRRHRPRSHLGQILRRRPWLVHPRHRYRWRRKNGIFNTRLSRTFGYTVKATTVRTPSWAVDMMRFNLDDFVPPGGGTNKISIPSEYYRIRKVKVESWPCSPITQGDRGVGSTAVILDDNFVPKVNAQTYDPYVNYSSRHTIPQPFSYHSRYFTPKPVLDSTIDYFQPNNKRNQLWLRLQTSRNVDHVGLGTAFENSIYDQDYNIRVTMYVQFREFNLKDPPLKP

SEQ ID NO: 4 - DNA, 1767 bp, Porcine Circovirus type 2
  CDS (51)..(992): GC0505 positive strand, replicase protein
accagcgcacttcggcagcggcagcacctcggcagcacctcagcagcaacatgcccagcaagaagaatggaagaagcggaccccaaccccataaaaggtgggtgttcactctgaataatccttccgaagacgagcgcaagaaaatacgggatcttccaatatccctgtttgattattttattgttggcgaggagggtaatgaggaaggacgaacacctcacctccaggggttcgctaattttgtgaagaagcagacttttaataaagtgaagtggtatttgggtgcccgctgccacatcgagaaagcgaaaggaacagatcagcagaataaagaatactgcagtaaagaaggcaacttactgatggagtgtggagctcctagatctcagggacaacggagtgacctgtctactgctgtgagtaccttgttggagagcgggagtctggtgaccgttgcagagcagcaccctgtaacgtttgtcagaaatttccgcgggctggctgaacttttgaaagtgagcgggaaaatgcagaagcgtgattggaagactaatgtacacgtcattgtggggccacctgggtgtggtaaaagcaaatgggctgctaattttgcagacccggaaaccacatactggaaaccacctagaaacaagtggtgggatggttaccatggtgaagaagtggttgttattgatgacttttatggctggctgccctgggatgatctactgagactgtgtgatcgatatccattgactgtagagactaaaggtggaactgtaccttttttggcccgcagtattctgattaccagcaatcagaccccgttggaatggtactcctcaactgctgtcccagctgtagaagctctttatcggaggattacttccttggtattttggaagaatgctacagaacaatccacggaggaagggggccagttcgtcaccctttcccccccatgccctgaatttccatatgaaataaattactgagtcttttttatcacttcgtaatggtttttattattcattaagggttaagtggggggtctttaagattaaattctctgaattgtacatacatggttacacggatattgtattcctggtcgtatatactgttttcgaacgcagtgccgaggcctacgtggtctacatttccagtagtttgtagtctcagccacagctgatttcttttgttgtttggttggaagtaatcaatagtggaatctaggacaggtttgggggtaaagtagcgggagtggtaggagaagggctgggttatggtatggcgggaggagtagtttacataggggtcataggtgagggctgtggcctttgttacaaagttatcatctagaataacagcactggagcccactcccctgtcaccgtgggtgatcggggagcagggccagaattcaaccttaacctttcttattctgtagtacccatagggcacagagcgggggtttgagccccctcctgggggaagaaagtcattaatattgaatctcatcatgtccaccgcccaggagggcgttttgactgtggttcgcttgatagtatatccgaaggtgcgggagaggcgggtgttgaagatgccatttttccttctccagcggtaacggtggcgggggtggacgagccaggggcggcggcggaggatctggccaagatggctgcgggggcggtgtcttcttctccggtaacgcctccttggatacgtcatatctgaaaacgaaagaagtgcgctgtaagtatt

SEQ ID NO: 5 - protein, 314 aa, Porcine Circovirus type 2
  GC0505
MPSKKNGRSGPQPHKRWVFTLNNPSEDERKKIRDLPISLFDYFIVGEEGNEEGRTPHLQGFANFVKKQTFNKVKWYLGARCHIEKAKGTDQQNKEYCSKEGNLLMECGAPRSQGQRSDLSTAVSTLLESGSLVTVAEQHPVTFVRNFRGLAELLKVSGKMQKRDWKTNVHVIVGPPGCGKSKWAANFADPETTYWKPPRNKWWDGYHGEEVVVIDDFYGWLPWDDLLRLCDRYPLTVETKGGTVPFLARSILITSNQTPLEWYSSTAVPAVEALYRRITSLVFWKNATEQSTEEGGQFVTLSPPCPEFPYEINY

SEQ ID NO: 6 - protein, 233 aa, Porcine Circovirus type 2
  GC0505
MTYPRRRYRRRRHRPRSHLGQILRRRPWLVHPRHRYRWRRKNGIFNTRLSRTFGYTIKRTTVKTPSWAVDMMRFNINDFLPPGGGSNPRSVPYGYYRIRKVKVEFWPCSPITHGDRGVGSSAVILDDNFVTKATALTYDPYVNYSSRHTITQPFSYHSRYFTPKPVLDSTIDYFQPNNKRNQLWLRLQTTGNVDHVGLGTAFENSIYDQEYNIRVTMYVQFREFNLKDPPLNP

SEQ ID NO: 7 - DNA, 20 bp, Artificial Sequence
  Forward primer
taggttagggctgtggcctt

SEQ ID NO: 8 - DNA, 20 bp, Artificial Sequence
  Reverse primer
ccgcaccttcggatatactg